SEQUENCE LISTING (74 sequences)

SEQ ID NO: 1 - protein, 258 aa, Artificial Sequence
  Synthetic amino acid sequence
CGSSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 2 - protein, 261 aa, Artificial Sequence
  Synthetic amino acid sequence
CGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 3 - protein, 4 aa, Artificial Sequence
  Synthetic amino acid sequence
CGSS

SEQ ID NO: 4 - protein, 12 aa, Artificial Sequence
  Synthetic amino acid sequence
CGSSGGGGVAAD

SEQ ID NO: 5 - protein, 15 aa, Artificial Sequence
  Synthetic amino acid sequence
CSSGSGSGGGGVAAD

SEQ ID NO: 6 - protein, 255 aa, Artificial Sequence
  Synthetic amino acid sequence
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVDLAAAYIKPDEKHHAVISGSVLYNQDEKGSYSLGIFGGKAEEVAGSAEVKTVNGIRHIGLAAKQ

SEQ ID NO: 7 - protein, 259 aa, Neisseria meningitidis (group B)
CSSGGGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGDKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 8 - protein, 261 aa, Neisseria meningitidis (group B)
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 9 - protein, 254 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 10 - protein, 254 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 11 - protein, 263 aa, Neisseria meningitidis (group B)
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFDKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 12 - protein, 255 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 13 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 14 - protein, 255 aa, Neisseria meningitidis (group B)
CSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 15 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 16 - protein, 263 aa, Neisseria meningitidis (group B)
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 17 - protein, 255 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 18 - protein, 254 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 19 - skipped

SEQ ID NO: 20 - protein, 257 aa, Neisseria meningitidis (group B)
CSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 21 - protein, 254 aa, Neisseria meningitidis (group B)
CSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 22 - protein, 262 aa, Neisseria meningitidis (group B)
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKIDSLINQRSFLVSGLGGEHTAFNQLPGDKAEYHGKAFSSDDPNGRLHYTIDFTNKQGYGRIEHLKTPELNVDLASAELKADEKSHAVILGDTRYGSEEKGTYHLALFGDRAQEIAGSATVKIGEKVHEIGIAGKQ

SEQ ID NO: 23 - protein, 255 aa, Neisseria meningitidis (group B)
CSSGGGGVAADIGAGLADALTAPLDHKDKGLQSLILDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFSSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 24 - protein, 17 aa, Artificial Sequence
  Synthetic amino acid sequence
CGSSGGGGSGGGGVTAD

SEQ ID NO: 25 - protein, 16 aa, Artificial sequence
  Synthetic amino acid sequence
CSSGGGGSGGGGVTAD

SEQ ID NO: 26 - protein, 255 aa, Neisseria meningitidis; identical to SEQ ID NO: 17

SEQ ID NO: 27 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 28 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 29 - protein, 260 aa, Neisseria meningitidis
CSSGGGGSGGGGVAADIGTGLADALTAPLDHKDKGLQSLMLDQSVRKNEKLKLSAQGAEKTYGNGDSLNTGKLKNDKISRFDFIHQIEVDGQLITLESGEFQVYKQSHSALTALQTEQVQDSEHSEKMVAKRRFKIGDIAGEHTSFDKLPKDVMATYRGTAFGSDDAGGKLTYTIDFAAKQGHGKIEHLKSPELNVELAAAYIKPDEKRHAVISGSVLYNQDEKGSYSLGIFGGQAQEVAGSAEVETANGIHHIGLAAKQ

SEQ ID NO: 30 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 31 - protein, 254 aa, Artificial Sequence
  Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 32 - protein, 253 aa, Artificial Sequence
  Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 33 - protein, 253 aa, Artificial Sequence
  Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 34 - protein, 256 aa, Artificial Sequence
  Synthetic amino acid sequence
SSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 35 - protein, 253 aa, Artificial Sequence
  Synthetic amino acid sequence
SSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 36 - protein, 261 aa, Artificial Sequence
  Synthetic amino acid sequence
SSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKIDSLINQRSFLVSGLGGEHTAFNQLPGDKAEYHGKAFSSDDPNGRLHYTIDFTNKQGYGRIEHLKTPELNVDLASAELKADEKSHAVILGDTRYGSEEKGTYHLALFGDRAQEIAGSATVKIGEKVHEIGIAGKQ

SEQ ID NO: 37 - protein, 254 aa, Artificial Sequence
  Synthetic amino acid sequence
SSGGGGVAADIGAGLADALTAPLDHKDKGLQSLILDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFSSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 38 - protein, 254 aa, Artificial Sequence; identical to SEQ ID NO: 31
  Synthetic amino acid sequence

SEQ ID NO: 39 - protein, 253 aa, Artificial Sequence
  Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 40 - protein, 253 aa, Artificial Sequence; identical to SEQ ID NO: 33
  Synthetic amino acid sequence

SEQ ID NO: 41 - protein, 259 aa, Artificial Sequence
  Synthetic amino acid sequence
SSGGGGSGGGGVAADIGTGLADALTAPLDHKDKGLQSLMLDQSVRKNEKLKLSAQGAEKTYGNGDSLNTGKLKNDKISRFDFIHQIEVDGQLITLESGEFQVYKQSHSALTALQTEQVQDSEHSEKMVAKRRFKIGDIAGEHTSFDKLPKDVMATYRGTAFGSDDAGGKLTYTIDFAAKQGHGKIEHLKSPELNVELAAAYIKPDEKRHAVISGSVLYNQDEKGSYSLGIFGGQAQEVAGSAEVETANGIHHIGLAAKQ

SEQ ID NO: 42 - protein, 254 aa, Artificial Sequence
  Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 43 - protein, 262 aa, Artificial Sequence
  Synthetic amino acid sequence
SSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 44 - protein, 254 aa, Artificial Sequence
  Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 45 - protein, 259 aa, Artificial Sequence
  Synthetic amino acid sequence
SSGGGGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 46 - protein, 260 aa, Artificial Sequence
  Synthetic amino acid sequence
SSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 47 - protein, 259 aa, Artificial Sequence
  Synthetic amino acid sequence
SSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 48 - protein, 260 aa, Artificial Sequence
  Synthetic amino acid sequence
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 49 - protein, 255 aa, Artificial Sequence; identical to SEQ ID NO: 23
  Synthetic amino acid sequence

SEQ ID NO: 50 - protein, 255 aa, Artificial Sequence; identical to SEQ ID NO: 17
  Synthetic amino acid sequence

SEQ ID NO: 51 - protein, 255 aa, Artificial Sequence
  Synthetic amino acid sequence
GSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 52 - protein, 254 aa, Artificial Sequence
  Synthetic amino acid sequence
GSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 53 - protein, 258 aa, Artificial Sequence
  Synthetic amino acid sequence
SSGGGGSGGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 54 - protein, 253 aa, Artificial Sequence
  Synthetic amino acid sequence
SSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 55 - protein, 255 aa, Artificial Sequence
  Synthetic amino acid sequence
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 56 - protein, 254 aa, Artificial Sequence
  Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 57 - protein, 262 aa, Artificial Sequence; identical to SEQ ID NO: 22
  Synthetic amino acid sequence

SEQ ID NO: 58 - protein, 254 aa, Artificial Sequence
  Synthetic amino acid sequence
SSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 59 - protein, 258 aa, Artificial Sequence; identical to SEQ ID NO: 1
  Synthetic amino acid sequence

SEQ ID NO: 60 - protein, 257 aa, Artificial Sequence
  Synthetic amino acid sequence
GSSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 61 - protein, 254 aa, Artificial Sequence
  Synthetic amino acid sequence
SSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 62 - protein, 252 aa, Artificial Sequence
  Synthetic amino acid sequence
GGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 63 - protein, 262 aa, Neisseria meningitidis (group B)
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 64 - protein, 262 aa, Neisseria meningitidis (group B)
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 65 - protein, 257 aa, Neisseria meningitidis (group B)
CSSGGGGVAADIGAGLADALTAPLDHKDKGLKSLTLEDSISQNGTLTLSAQGAERTFKAGNKDNSLNTGKLKNDKISRFDFVQKIEVDGQTITLASGEFQIYKQNHSAVVALQIEKINNPDKIDSLINQRSFLVSSLGGEHTAFNQLPGGKAEYHGKAFSSDDPNGRLHYSIDFTKKQGYGRIEHLKTPEQNVELAAAELKADEKSHAVILGDTRYGSEEKGTYHLALFGDRAQEIAGSATVKIGEKVHEIGIAGKQ

SEQ ID NO: 66 - protein, 262 aa, Neisseria meningitidis (group B)
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYDSEEKGTYHLALFGDRAQEIAGSATVKIGEKVHEISIAGKQ

SEQ ID NO: 67 - protein, 262 aa, Neisseria meningitidis (group B)
CSSGGGGSGGGGVAADIGAGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQTEKVNNPDKTDSLINQRSFLVSGLGGEHTAFNQLPVGKSEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 68 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFSSDDAGGKLTYTIDFAAKQGHGKIEHLKSPELNVELATAYIKPDEKRHAVISGSVLYNQDEKGSYSLGIFGGQAQEVAGSAEVETANGIQHIGLAAKQ

SEQ ID NO: 69 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTALGSDDAGGKLTYTIDFAAKQGHGKIEHLKSPELNVELATAYIKPDEKRHAVISGSVLYNQDEKGSYSLGIFGGQAQEVAGSAEVETVNGIHHIGLAAKQ

SEQ ID NO: 70 - protein, 255 aa, Neisseria meningitidis (group B)
CSSGGGGVAADIGAGLADALTAPLDHKDKSLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 71 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDRQLITLESGEFQVYKQSHSALTALQTEQVQDSEHSGKMVAKRQFRIGDIAGEHTSFDKLPEGGRATYRGTAFGSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 72 - protein, 255 aa, Neisseria meningitidis (group B)
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDLEHSGKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 73 - protein, 258 aa, Neisseria meningitidis (group B)
CSSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 74 - protein, 257 aa, Neisseria meningitidis (group B)
CSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

(19) United States
(12) Patent Application Publication
(10) Pub. No.: US 2022/0118072 A1
(43) Pub. Date: Apr. 21, 2022

(54) NEISSERIA MENINGITIDIS COMPOSITIONS AND METHODS THEREOF

(71) Applicant: Pfizer Inc., New York, NY (US)

(72) Inventors: Annaliesa Sybil Anderson, Upper Saddle River, NJ (US); Paul Liberator, Holmdel, NJ (US); Thomas Richard Jones, Bluffton, SC (US); Kathrin Ute Jansen, New York, NY (US); John Lance Perez, Doylestown, PA (US); Shannon Lea Harris, Boston, MA (US)

(21) Appl. No.: 17/428,487

(22) PCT Filed: Feb. 7, 2020
(86) PCT No.: PCT/IB2020/050988

§ 371 Date: Aug. 4, 2021

Related U.S. Application Data
(60) Provisional application No. 62/803,730, filed on Feb. 11, 2019.
(60) Provisional application No. 62/869,423, filed on Jul. 1, 2019.

(51) Int. Cl. A61K 39/095; A61K 47/26; A61K 47/22; A61K 47/02

(52) U.S. Cl. CPC A61K 39/095; A61K 47/26; A61K 2039/55505; A61K 47/02; A61K 47/22

(57) ABSTRACT

In one aspect, the invention relates to use of a composition including a first polypeptide and a second polypeptide, wherein the composition elicits an immune response against Neisseria meningitis serogroup B strains expressing, for example, variants A02, A28, A42, A63, A76, B05, B07, B08, B13, B52 and B107.

### CROSS REFERENCE TO RELATED APPLICATIONS

The present application claims the benefit of U.S. Provisional Patent Application 62/803,730, filed Feb. 11, 2019 and U.S. Provisional Patent Application 62/869,423, filed Jul. 1, 2019. Each of the foregoing applications are hereby incorporated by reference in their entireties.

### FIELD OF THE INVENTION

The present invention relates to Neisseria meningitidis compositions and methods thereof.

### BACKGROUND OF THE INVENTION

Neisseria meningitidis is a Gram-negative encapsulated bacterium that can cause sepsis, meningitis, and death. N. meningitidis can be classified into at least 12 serogroups (including serogroups A, B, C, 29E, H, I, K, L, W, X, Y and Z) based on chemically and antigenically distinctive polysaccharide capsules. Strains representative of five of the serogroups (A, B, C, Y, and W) are responsible for the majority of disease.

Meningococcal meningitis is a devastating disease that can kill children and young adults within hours despite the availability of antibiotics.

TRUMENBA (bivalent rLP2086), a vaccine for the prevention of Neisseria meningitis serogroup B (MenB) disease, consists of two protein antigens, variants of meningococcal factor H binding protein (fHBP). fHBP exists as two subfamilies, A and B. Within each subfamily several hundred unique fHBP variants have been identified. Despite this sequence diversity, a vaccine containing one protein from each subfamily was demonstrated to induce broad coverage across MenB strains that represent the diversity of fHBP variants. Licensure was based on the ability of the vaccine to elicit antibodies that initiate complement-mediated killing of invasive MenB strains in a serum bactericidal assay using human complement (hSBA). Due to the endemic nature of meningococcal disease, it is not possible to predict which fHBP variants individuals may be exposed to.

Accordingly, continuing to explore the coverage conferred by a vaccine for the prevention of Neisseria meningitis serogroup B (MenB) disease is useful and helpful to provide additional evidence to illustrate the breadth of immune coverage.

### SUMMARY OF THE INVENTION

In one aspect, the invention relates to uses of a composition including a first lipidated polypeptide variant of a Neisseria meningitidis serogroup B factor H binding protein (fHBP) and a second lipidated polypeptide variant of a Neisseria meningitidis serogroup B fHBP. In one embodiment, the composition induces a bactericidal immune response against at least one N. meningitidis serogroup B strain expressing a polypeptide selected from the group consisting of A02, A28, A42, A63, A76, B05, B07, B08, B13, B52 and B107.

For example, in one aspect, the invention relates to uses of a composition including a first lipidated polypeptide including the amino acid sequence set forth in SEQ ID NO: 1 and a second lipidated polypeptide including the amino acid sequence set forth in SEQ ID NO: 2.

In one aspect, the invention relates to use of an effective amount of a composition for inducing a bactericidal immune response against a Neisseria meningitidis serogroup B strain, including a subfamily A strain and a subfamily B strain.

In another aspect, the invention relates to use of an effective amount of a composition for inducing a bactericidal immune response against a Neisseria meningitidis serogroup B subfamily B strain in a human. In a preferred aspect, the invention relates to use of an effective amount of a composition for inducing a bactericidal immune response against a Neisseria meningitidis serogroup B subfamily A strain and against a Neisseria meningitidis serogroup B subfamily B strain in a human. The use includes administering to the human an effective amount of a composition.

In one embodiment, the composition includes a) a first lipidated polypeptide comprising the amino acid sequence set forth in SEQ ID NO: 1, and b) a second lipidated polypeptide comprising the amino acid sequence set forth in SEQ ID NO: 2. In one embodiment, the composition induces a bactericidal immune response against at least one N. meningitidis serogroup B strain expressing a polypeptide selected from the group consisting of A02, A28, A42, A63, A76, B05, B07, B08, B13, B52 and B107.

In one embodiment, the composition further includes polysorbate-80. In one embodiment, the composition further includes aluminum. In one embodiment, the composition further includes histidine buffer. In one embodiment, the composition further includes sodium chloride. In one embodiment, the composition includes about 120 μg/ml of the first polypeptide; about 120 μg/ml of the second polypeptide; about 2.8 molar ratio of polysorbate-80; about 0.5 mg/ml aluminum; about 10 mM histidine; and about 150 mM sodium chloride. In one embodiment, the composition includes about 60 μg of the first polypeptide; about 60 μg of the second polypeptide; about 18 μg polysorbate-80; about 250 μg aluminum; about 780 μg histidine; and about 4380 μg sodium chloride.

In one embodiment, the composition further includes at least one additional immunogenic composition comprising a mixture of four distinct and separately made protein-capsular polysaccharide conjugates, wherein the first conjugate includes N. meningitidis capsular polysaccharide of serogroup W conjugated to a carrier protein, the second conjugate includes N. meningitidis capsular polysaccharide of serogroup Y conjugated to a carrier protein, the third conjugate includes N. meningitidis capsular polysaccharide of serogroup A conjugated to a carrier protein, and the fourth conjugate includes N. meningitidis capsular polysaccharide of serogroup C conjugated to a carrier protein, wherein the carrier protein is selected from the group consisting of diphtheria toxoid, CRM₁₉₇, and tetanus toxoid. In one embodiment, the carrier protein is diphtheria toxoid. In one embodiment, the carrier protein is tetanus toxoid. In one embodiment, the at least one additional immunogenic composition is a liquid composition. In one embodiment, the at least one additional immunogenic composition is not lyophilized. In one embodiment, the use includes inducing an immune response against at least one of a Neisseria meningitidis serogroup A strain, a Neisseria meningitidis serogroup C strain, a Neisseria meningitidis serogroup Y strain, and/or a Neisseria meningitidis serogroup W strain, or any combination thereof.

In one embodiment, the Neisseria meningitidis serogroup A (MenA) capsular saccharide is conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); the Neisseria meningitidis serogroup C (MenC) capsular saccharide is conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); the Neisseria meningitidis serogroup W (MenW) capsular saccharide is directly conjugated to tetanus toxoid carrier protein (TT) by 1-cyano-4-dimethylamino pyridinium tetrafluoroborate chemistry, in the absence of a linker (MenW-TT conjugate); and the Neisseria meningitidis serogroup Y (MenY) capsular saccharide is directly conjugated to tetanus toxoid carrier protein (TT) by 1-cyano-4-dimethylamino pyridinium tetrafluoroborate chemistry, in the absence of a linker (MenY-TT conjugate). In one embodiment, the composition does not include a MenA capsular saccharide in the absence of an adipic acid dihydrazide (ADH) linker.

In one embodiment, the effective amount of the composition includes one dose. In one embodiment, the effective amount of the composition includes two doses. In one embodiment, the effective amount of the composition further includes a booster dose. In one embodiment, the effective amount of the composition includes at most two doses. In one embodiment, the effective amount of the composition includes at most three doses.

In one embodiment, the composition does not include a hybrid protein. In one embodiment, the composition does not include a fusion protein. In one embodiment, the composition is not lyophilized. In one embodiment, the composition does not include formaldehyde. In one embodiment, the composition does not include diphtheria toxoid or CRM.

In one embodiment, the patient is aged 12 to <18 Months or 18 to <24 Months. In one embodiment, the patient is aged 18 to <24 Months. In one embodiment, the patient is aged 24 Months to <10 Years.

In one embodiment, the composition induces a bactericidal titer of serum immunoglobulin that is at least 2-fold higher in the human after receiving the first dose than a bactericidal titer of serum immunoglobulin in the human prior to receiving the first dose, when measured under identical conditions in a serum bactericidal assay using human complement.

In one embodiment, the composition induces a bactericidal titer of serum immunoglobulin that is at least 4-fold higher in the human after receiving the first dose than a bactericidal titer of serum immunoglobulin in the human prior to receiving the first dose, when measured under identical conditions in a serum bactericidal assay using human complement.

In one embodiment, the composition induces a bactericidal titer of serum immunoglobulin that is at least 8-fold higher in the human after receiving the first dose than a bactericidal titer of serum immunoglobulin in the human prior to receiving the first dose, when measured under identical conditions in a serum bactericidal assay using human complement.

### BRIEF DESCRIPTION OF THE DRAWINGS

FIG. 1A Factor H binding protein (FHbp) phylogenetic tree: primary and additional Neisseria meningitidis serogroup B (MenB) test strain variants and variant prevalence of primary and additional MenB test strains. In FIG. 1A, the phylogenetic and FHbp subfamily relationship of the FHbp variants expressed by the four primary and 10 additional MenB test strains is illustrated. The scale bar indicates genetic distance based on protein sequence. The amino acid sequence identity within FHbp subfamilies is ≥83%33. hSBA=serum bactericidal assay using human complement.

FIG. 1B depicts variant prevalence (left vertical axis; bars) and cumulative prevalence (right vertical axis; circles) based on the MenB isolate collection (n=1263). Variants are ordered based on their prevalence rank in the MenB isolate collection. Note that scales are different between left and right y-axes.

FIG. 2 illustrates an algorithm used for the selection of additional Neisseria meningitidis Serogroup B (MenB) test strains. The MenB isolate collection (n=1263) is used as the example in this figure. FHbp factor H binding protein, ST sequence type.

FIG. 3 depicts a graph, wherein the diamonds (“Killed”) mark those strains that were susceptible in hSBAs. A strain was considered susceptible to the composition (which includes a first polypeptide having SEQ ID NO: 1 and a second polypeptide having SEQ ID NO: 2 (i.e., TRUMENBA)) immune sera if a 4-fold rise in the hSBA titer was achieved between the pre- and post-vaccination serum samples. Dark triangles (“Not Killed”) correspond to strains that did not achieve a 4-fold rise in hSBA titer from baseline. The eleven fHBP variants disclosed herein (i.e., A02, A28, A42, A63, A76, B05, B07, B08, B13, B52 and B107) were each represented by one strain in this study and each is susceptible to TRUMENBA immune sera in hSBAs. These strains are annotated in FIG. 3 and detailed in Table 1. The 109 MenB strains evaluated in this study are ordered from high to low fHBP surface expression levels determined using the MEASURE assay. Each strain was also tested in the hSBA using pools of subject matched pre- and post-vaccination serum samples (prior to vaccination and 1 month following a third dose of TRUMENBA).

### SEQUENCE IDENTIFIERS

SEQ ID NO: 1 sets forth the amino acid sequence for a recombinant N. meningitidis, serogroup B, 2086 variant A05 polypeptide antigen.
SEQ ID NO: 2 sets forth the amino acid sequence for a recombinant N. meningitidis, serogroup B, 2086 variant B01 polypeptide antigen.
SEQ ID NO: 3 sets forth the amino acid residues at positions 1˜4 of SEQ ID NO: 1 and SEQ ID NO: 2.
SEQ ID NO: 4 sets forth the amino acid sequence of the N-terminus of a recombinant Neisserial Subfamily A LP2086 polypeptide (rLP2086) (A05) polypeptide.
SEQ ID NO: 5 sets forth the amino acid sequence of the N-terminus of Neisserial Subfamily A LP2086 M98250771 polypeptide (A05) polypeptide.
SEQ ID NO: 6 sets forth the amino acid sequence for N. meningitidis, serogroup B, 2086 variant B153.
SEQ ID NO: 7 sets forth the amino acid sequence for N. meningitidis, serogroup B, 2086 variant A04.
SEQ ID NO: 8 sets forth the amino acid sequence for N. meningitidis, serogroup B, 2086 variant A05 SEQ ID NO: 9 sets forth the amino acid sequence for N. meningitidis, serogroup B, 2086 variant A12.
SEQ ID NO: 10 sets forth the amino acid sequence for N. meningitidis, serogroup B, 2086 variant A22.
SEQ ID NO: 11 sets forth the amino acid sequence for N. meningitidis, serogroup B, 2086 variant B02.
SEQ ID NO: 12 sets forth the amino acid sequence for N. meningitidis, serogroup B, 2086 variant B03.
SEQ ID NO: 13 sets forth the amino acid sequence for N. meningitidis, serogroup B, 2086 variant B09.
SEQ ID NO: 14 sets forth the amino acid sequence for N. meningitidis, serogroup B, 2086 variant B22.
SEQ ID NO: 15 sets forth the amino acid sequence for N. meningitidis, serogroup B, 2086 variant B24.
SEQ ID NO: 16 sets forth the amino acid sequence for N. meningitidis, serogroup B, 2086 variant B44.
SEQ ID NO: 17 sets forth the amino acid sequence for N. meningitidis, serogroup B, 2086 variant B16.
SEQ ID NO: 18 sets forth the amino acid sequence for N. meningitidis, serogroup B, 2086 variant A07.
SEQ ID NO: 19 sets forth the amino acid sequence for N. meningitidis, serogroup B, 2086 variant A19.
SEQ ID NO: 20 sets forth the amino acid sequence for N. meningitidis, serogroup B, 2086 variant A06.
SEQ ID NO: 21 sets forth the amino acid sequence for N. meningitidis, serogroup B, 2086 variant A15.
SEQ ID NO: 22 sets forth the amino acid sequence for N. meningitidis, serogroup B, 2086 variant A29.
SEQ ID NO: 23 sets forth the amino acid sequence for N. meningitidis, serogroup B, 2086 variant B15.
SEQ ID NO: 24 sets forth the amino acid sequence of the N-terminus of a recombinant Neisserial Subfamily B LP2086 polypeptide (rLP2086) (B01) polypeptide.
SEQ ID NO: 25 sets forth the amino acid sequence of the N-terminus of Neisserial Subfamily B LP2086 CDC-1573 polypeptide (B01) polypeptide.
SEQ ID NO: 26 sets forth the amino acid sequence for N. meningitidis serogroup A strain expressing factor H binding protein (fHBP) B16.
SEQ ID NO: 27 sets forth the amino acid sequence for a N. meningitidis serogroup C strain expressing fHBP A10. SEQ ID NO: 27 also sets forth the amino acid sequence for a N. meningitidis serogroup W strain expressing fHBP A10.
SEQ ID NO: 28 sets forth the amino acid sequence for a N. meningitidis serogroup W strain expressing fHBP A19.
SEQ ID NO: 29 sets forth the amino acid sequence for a N. meningitidis serogroup Y strain expressing fHBP B47.
SEQ ID NO: 30 sets forth the amino acid sequence for a N. meningitidis serogroup X strain expressing fHBP B49.
SEQ ID NO: 31 sets forth the amino acid sequence for a non-lipidated N. meningitidis, serogroup B, 2086 variant B16.
SEQ ID NO: 32 sets forth the amino acid sequence for a non-lipidated N. meningitidis, serogroup B, 2086 variant A07.
SEQ ID NO: 33 sets forth the amino acid sequence for a non-lipidated N. meningitidis, serogroup B, 2086 variant A19.
SEQ ID NO: 34 sets forth the amino acid sequence for a non-lipidated N. meningitidis, serogroup B, 2086 variant A06.
SEQ ID NO: 35 sets forth the amino acid sequence for a non-lipidated N. meningitidis, serogroup B, 2086 variant A15.
SEQ ID NO: 36 sets forth the amino acid sequence for a non-lipidated N. meningitidis, serogroup B, 2086 variant A29.
SEQ ID NO: 37 sets forth the amino acid sequence for a non-lipidated N. meningitidis, serogroup B, 2086 variant B15.
SEQ ID NO: 38 sets forth the amino acid sequence for a non-lipidated N. meningitidis serogroup A strain expressing factor H binding protein (fHBP) B16.
SEQ ID NO: 39 sets forth the amino acid sequence for a non-lipidated N. meningitidis serogroup C strain expressing fHBP A10. SEQ ID NO: 39 also sets forth the amino acid sequence for a non-lipidated N. meningitidis serogroup W strain expressing fHBP A10.
SEQ ID NO: 40 sets forth the amino acid sequence for a non-lipidated N. meningitidis serogroup W strain expressing fHBP A19.
SEQ ID NO: 41 sets forth the amino acid sequence for a non-lipidated N. meningitidis serogroup Y strain expressing fHBP B47.
SEQ ID NO: 42 sets forth the amino acid sequence for a non-lipidated N. meningitidis serogroup X strain expressing fHBP B49.
SEQ ID NO: 43 sets forth the amino acid sequence for a non-lipidated N. meningitidis, serogroup B, 2086 variant B44.
SEQ ID NO: 44 sets forth the amino acid sequence for a non-lipidated N. meningitidis, serogroup B, 2086 variant B09.
SEQ ID NO: 45 sets forth the amino acid sequence for a N. meningitidis, serogroup B, 2086 variant B09.
SEQ ID NO: 46 sets forth the amino acid sequence for a non-lipidated N. meningitidis, serogroup B, 2086 variant A05.
SEQ ID NO: 47 sets forth the amino acid sequence for a non-lipidated N. meningitidis, serogroup B, 2086 variant B01.
SEQ ID NO: 48 sets forth the amino acid sequence for a N. meningitidis, serogroup B, 2086 variant B01, which includes an N-terminal Cys at amino acid position 1.
SEQ ID NO: 49 sets forth the amino acid sequence for a N. meningitidis, serogroup B, 2086 variant B15, which includes an N-terminal Cys at amino acid position 1.
SEQ ID NO: 50 sets forth the amino acid sequence for a N. meningitidis, serogroup B, 2086 variant B16, which includes an N-terminal Cys at amino acid position 1.
SEQ ID NO: 51 sets forth the amino acid sequence for a N. meningitidis, serogroup B, 2086 variant B22.
SEQ ID NO: 52 sets forth the amino acid sequence for a N. meningitidis, serogroup B, 2086 variant A22.
SEQ ID NO: 53 sets forth the amino acid sequence for a non-lipidated N. meningitidis, serogroup B, 2086 variant A12.
SEQ ID NO: 54 sets forth the amino acid sequence for a non-lipidated N. meningitidis, serogroup B, 2086 variant A22.
SEQ ID NO: 55 sets forth the amino acid sequence for a N. meningitidis serogroup B, 2086 variant A62, which includes an N-terminal Cys at amino acid position 1.
SEQ ID NO: 56 sets forth the amino acid sequence for a non-lipidated N. meningitidis, serogroup B, 2086 variant A62.
SEQ ID NO: 57 sets forth the amino acid sequence for a N. meningitidis, serogroup B, 2086 variant A29, which includes an N-terminal Cys at amino acid position 1.
SEQ ID NO: 58 sets forth the amino acid sequence for a non-lipidated N. meningitidis, serogroup B, 2086 variant B22.
SEQ ID NO: 59 sets forth the amino acid sequence for a N. meningitidis, serogroup B, 2086 variant A05.
SEQ ID NO: 60 sets forth the amino acid sequence for a non-lipidated N. meningitidis, serogroup B, 2086 variant A05.
SEQ ID NO: 61 sets forth the amino acid sequence for a N. meningitidis, serogroup B, 2086 variant B24.
SEQ ID NO: 62 sets forth the amino acid sequence for a N. meningitidis, serogroup B, 2086 variant B24.
SEQ ID NO: 63 sets forth the amino acid sequence for a N. meningitidis, serogroup B, 2086 variant A02 SEQ ID NO: 64 sets forth the amino acid sequence for a N. meningitidis, serogroup B, 2086 variant A28.
SEQ ID NO: 65 sets forth the amino acid sequence for a N. meningitidis, serogroup B, 2086 variant A42.
SEQ ID NO: 66 sets forth the amino acid sequence for a N. meningitidis, serogroup B, 2086 variant A63.
SEQ ID NO: 67 sets forth the amino acid sequence for a N. meningitidis, serogroup B, 2086 variant A76.
SEQ ID NO: 68 sets forth the amino acid sequence for a N. meningitidis, serogroup B, 2086 variant B05.
SEQ ID NO: 69 sets forth the amino acid sequence for a N. meningitidis, serogroup B, 2086 variant B07.
SEQ ID NO: 70 sets forth the amino acid sequence for a N. meningitidis, serogroup B, 2086 variant B08.
SEQ ID NO: 71 sets forth the amino acid sequence for a N. meningitidis, serogroup B, 2086 variant B13.
SEQ ID NO: 72 sets forth the amino acid sequence for a N. meningitidis, serogroup B, 2086 variant B52.
SEQ ID NO: 73 sets forth the amino acid sequence for a N. meningitidis, serogroup B, 2086 variant B107.
SEQ ID NO: 74 sets forth the amino acid sequence for a N. meningitidis, serogroup B, 2086 variant A56.

### DETAILED DESCRIPTION OF THE INVENTION

The inventors surprisingly discovered additional medical uses for a composition that includes a first lipidated polypeptide and a second lipidated polypeptide. For example, disclosed herein are methods of inducing an immune response against at least one Neisseria meningitidis serogroup B strain, wherein the strain expresses any one factor H binding protein (fHBP) variant selected from A02, A28, A42, A63, A76, B05, B07, B08, B13, B52 and B107, in a mammal by administering a composition that includes a first lipidated polypeptide and a second lipidated polypeptide. An exemplary polypeptide in the composition may include a polypeptide having any one sequence set forth in SEQ ID NO: 1, SEQ ID NO: 2, and SEQ ID NO: 6-74.

In a preferred embodiment, the composition includes a first lipidated polypeptide including the amino acid sequence set forth in SEQ ID NO: 1 and a second lipidated polypeptide including the amino acid sequence set forth in SEQ ID NO: 2. The composition has an acceptable safety profile in humans and the composition surprisingly elicits a broadly cross-reactive bactericidal immune response in humans against at least one Neisseria meningitidis strain or strains selected from the group consisting of strains expressing variants A02, A28, A42, A63, A76, B05, B07, B08, B13, B52 and B107.

### Composition and Vaccine

In one aspect, the invention relates to uses of a composition against Neisseria meningitidis. The composition includes a first lipidated polypeptide and a second lipidated polypeptide. An exemplary polypeptide in the composition may include a polypeptide having any one sequence selected from the sequences set forth in SEQ ID NO: 1, SEQ ID NO: 2, and SEQ ID NO: 6-74.

In a preferred embodiment, the composition includes a first lipidated polypeptide having the amino acid sequence set forth in SEQ ID NO: 1, and a second lipidated polypeptide having the amino acid sequence set forth in SEQ ID NO: 2.

In one embodiment, the composition does not include a fusion protein. In one embodiment, the composition does not include a chimeric protein. In one embodiment, the composition does not include a hybrid protein. In one embodiment, the composition does not further include a peptide fragment. In another embodiment, the composition does not further include a Neisserial polypeptide that is not fHBP. For example, in one embodiment, the composition does not include a PorA protein. In another embodiment, the composition does not include a NadA protein. In another embodiment, the composition does not further include a Neisserial heparin binding antigen (NHBA). In another embodiment, the composition does not further include a Neisserial outer membrane vesicle (OMV). In a preferred embodiment, the composition does not further include antigens, other than the first polypeptide and the second polypeptide.

In one embodiment, the composition includes additional polypeptides, such as, for example, any one of the following polypeptides: A02, A28, A42, A63, A76, B24, B16, B44, A22, B03, B09, A12, A19, A05, A07, A06, A15, A29, B01, A56, A62, B15, and any combination thereof. Preferably, the composition includes a combination of A05 and B01 polypeptides. In another preferred embodiment, the composition includes a combination of B24 and A05 polypeptides. In another embodiment, the composition includes a combination of A05, A12, B09, and B44 polypeptides. In one embodiment, the composition includes a lipidated fHBP. In one embodiment, the composition does not include a non-lipidated fHBP.

In another embodiment, the composition includes a non-lipidated fHBP, such as any one of the non-lipidated fHBP described in International Patent Publication No. WO2012/032489, US Patent Publication No. US20120093852, International Patent Publication No. WO2013/132452, and US Patent Publication No. US20160030543, which are each incorporated herein by reference in their entirety. In one embodiment, the composition includes at least one non-lipidated fHBP and at least one lipidated fHBP.

In some embodiments, the composition includes a polypeptide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identity to the amino acid sequence set forth in any one of SEQ ID NO: 1, SEQ ID NO: 2, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, and SEQ ID NO: 62.

In one aspect, the inventors surprisingly discovered that the polypeptide antigens induce an immune response against at least one strain of N. meningitidis serogroup B, such as at least one N. meningitidis serogroup B strain selected from the group consisting of A02, A28, A42, A63, A76, B05, B07, B08, B13, B52 and B107.

In one embodiment, the composition does not further include a polypeptide that is not derived from N. meningitidis serogroup B subfamily A M98250771 strain and/or N. meningitidis serogroup B subfamily B CDC1573 strain.

In one embodiment, the composition does not further include a polypeptide having less than 100% sequence identity to SEQ ID NO: 1. In another embodiment, the composition does not further include a polypeptide having less than 100% sequence identity to SEQ ID NO: 2. For example, the composition does not further include a polypeptide having less than 100% sequence identity to the full length of SEQ ID NO: 1 and/or SEQ ID NO: 2.

In one embodiment, the composition further includes polysorbate-80, aluminum, histidine, and sodium chloride. In one embodiment, the composition includes about 60 μg of a first lipidated polypeptide including the amino acid sequence set forth in SEQ ID NO: 1, about 60 μg of a second lipidated polypeptide including the amino acid sequence set forth in SEQ ID NO: 2, 2.8 molar ratio of polysorbate-80 to each polypeptide, 0.5 mg aluminum/ml as aluminum phosphate, 10 mM histidine, and 150 mM sodium chloride, wherein the composition preferably has a total volume of about 0.5 ml.

In another embodiment, the composition includes about 120 μg/ml of a first lipidated polypeptide including the amino acid sequence set forth in SEQ ID NO: 1, about 120 μg/ml of a second lipidated polypeptide including the amino acid sequence set forth in SEQ ID NO: 2, 2.8 molar ratio of polysorbate-80 to each polypeptide, 0.5 mg aluminum/ml as aluminum phosphate, 10 mM histidine, and 150 mM sodium chloride.

In a further embodiment, the composition includes a) 60 μg of a first lipidated polypeptide including the amino acid sequence set forth in SEQ ID NO: 1; b) 60 μg of a second lipidated polypeptide including the amino acid sequence set forth in SEQ ID NO: 2; c) 18 μg polysorbate-80; d) 250 μg aluminum; e) 780 μg histidine, and; 0 4380 μg sodium chloride.

In an exemplary embodiment, the composition includes about 60 μg of a first lipidated polypeptide consisting of the amino acid sequence set forth in SEQ ID NO: 1, about 60 μg of a second lipidated polypeptide consisting of the amino acid sequence set forth in SEQ ID NO: 2, 2.8 molar ratio of polysorbate-80 to first lipidated polypeptide and to second lipidated polypeptide, 0.5 mg/ml aluminum phosphate, 10 mM histidine, and 150 mM sodium chloride, wherein the composition has a total volume of about 0.5 ml. In the exemplary embodiment, the composition is a sterile isotonic buffered liquid suspension. In the exemplary embodiment, the composition has a pH 6.0. In the exemplary embodiment, the first polypeptide and the second polypeptide are adsorbed to aluminum.

In one embodiment, the composition has a total volume of about 0.5 ml. In one embodiment, a first dose of the composition has a total volume of about 0.5 ml. A “first dose” refers to the dose of the composition that is administered on Day 0. A “second dose” or “third dose” refers to the dose of the composition that is administered subsequent to the first dose, which may or may not be the same amount as the first dose.

The composition is immunogenic after administration of a first dose to a human. In one embodiment, the first dose is about 0.5 ml in total volume.

The composition induces a bactericidal titer of serum immunoglobulin against N. meningitidis serogroup B that is at least greater than 1-fold higher, preferably at least 2-fold higher, in the human after receiving the first dose than a bactericidal titer of serum immunoglobulin against N. meningitidis serogroup B in the human prior to receiving the first dose, when measured under identical conditions in a serum bactericidal assay using human complement (hSBA).

In a preferred embodiment, the bactericidal titer or bactericidal immune response is against a N. meningitidis serogroup B subfamily A strain and against a N. meningitidis serogroup B subfamily B strain. In another embodiment, the bactericidal titer or bactericidal immune response is against a N. meningitidis serogroup B subfamily A, A05 strain. In another embodiment, the bactericidal titer or bactericidal immune response is against a N. meningitidis serogroup B subfamily B, B01 strain. Most preferably, the bactericidal titer or bactericidal immune response is at least against N. meningitidis serogroup B, subfamily B, B01 strain and at least against N. meningitidis serogroup B, subfamily A, A05 strain.

In another preferred embodiment, the bactericidal titer or bactericidal immune response is at least against N. meningitidis serogroup B, subfamily B, B24 strain. In another preferred embodiment, the bactericidal titer or bactericidal immune response is at least against N. meningitidis serogroup B, subfamily A, A22 strain.

In one embodiment, the composition induces a bactericidal titer of serum immunoglobulin against N. meningitidis that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 16-fold higher in the human after receiving a dose of the composition than a bactericidal titer of serum immunoglobulin in the human prior to receiving said dose, when measured under identical conditions in a serum bactericidal assay using human complement.

In one embodiment, the composition induces a bactericidal titer of serum immunoglobulin against N. meningitidis serogroup B that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 16-fold higher in the human after receiving a dose of the composition than a bactericidal titer of serum immunoglobulin against N. meningitidis serogroup B in the human prior to receiving said dose, when measured under identical conditions in a serum bactericidal assay using human complement.

In one embodiment, the composition is an immunogenic composition for a human. In another embodiment, the composition is a vaccine. A “vaccine” refers to a composition that includes an antigen, which contains at least one epitope that induces an immune response that is specific for that antigen. The vaccine may be administered directly into the subject by subcutaneous, oral, oronasal, or intranasal routes of administration. Preferably, the vaccine is administered intramuscularly. In one embodiment, the composition is a vaccine for humans. In one embodiment, the composition is an immunogenic composition against N. meningitidis.

In one embodiment, the composition is a liquid composition. In a preferred embodiment, the composition is a liquid suspension composition. In another preferred embodiment, the composition is not lyophilized.

In one embodiment, the composition that includes a combination of a MenB bivalent rLP2086 composition and a MenACWY-TT composition. The MenB bivalent rLP2086 composition refers to a composition that includes a single N. meningitidis polypeptide component that induces an effective broadly protective immune response against multiple strains of N. meningitidis serogroup B. Specifically, in one embodiment, the MenB bivalent rLP2086 composition includes (a) a MenB rLP2086 subfamily A protein (SEQ ID NO: 1) and (b) MenB rLP2086 subfamily B protein (SEQ ID NO: 2).

The MenACWY-TT composition refers to a composition that includes purified capsular polysaccharides of Neisseria meningitidis Serogroups A, C, W and Y, each independently conjugated to TT at ratios (TT to polysaccharide) of ˜3, ˜3, ˜1.5 and ˜1.3, respectively. Specifically, the composition includes (c) a Neisseria meningitidis serogroup A (MenA) capsular saccharide conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); (d) a Neisseria meningitidis serogroup C (MenC) capsular saccharide conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); (e) a Neisseria meningitidis serogroup W (MenW) capsular saccharide directly conjugated to tetanus toxoid carrier protein (TT) by 1-cyano-4-dimethylamino pyridinium tetrafluoroborate chemistry, in the absence of a linker (MenW-TT conjugate); (f) a Neisseria meningitidis serogroup Y (MenY) capsular saccharide directly conjugated to tetanus toxoid carrier protein (TT) by 1-cyano-4-dimethylamino pyridinium tetrafluoroborate chemistry, in the absence of a linker (MenY-TT conjugate). Preferably, the MenACWY-TT composition is presented as a lyophilized powder.

MenA_{AH}-TT, MenC_{AH}-TT, MenW-TT, and MenY-TT conjugates are prepared through the following steps: manufacture of the polysaccharide drug substance intermediate, manufacture of the TT drug substance intermediate, microfluidization of the polysaccharide, derivatization of the polysaccharide (for the MenA_{AH}-TT and MenC_{AH}-TT processes only), additional purification of the TT, and conjugation of the individual polysaccharides to TT.

Regarding the MenA_{AH}-TT conjugate, the MenA polysaccharide is first microfluidized to reduce molecular size and viscosity, then activated via cyanylation with 1-cyano-4-dimethylamino-pyridinium tetrafluoroborate (CDAP). Activated MenA is derivatized with adipic acid dihydrazide (ADH) to form the MenA_{AH}. MenA_{AH} and Tetanus Toxoid (TT) are coupled through carbodiimide-mediated condensation (1-ethyl-3-(3-dimethylaminopropyl)carbodiimide (EDAC) coupling technology) to form MenA_{AH}-Tetanus Toxoid Conjugate (MenA_{AH}-TT).

Regarding the MenC_{AH}-TT conjugate, the MenC polysaccharide is first microfluidized to reduce molecular size and viscosity, then activated via cyanylation with CDAP. Activated MenC is derivatized with adipic acid dihydrazide (ADH) to form the MenC_{AH}. MenC_{AH} and TT are coupled through carbodiimide-mediated condensation EDAC coupling technology) to form MenC_{AH}-Tetanus Toxoid (MenC_{AH}-TT).

Regarding the MenW-TT conjugate, MenW polysaccharide is first microfluidized to reduce molecular size and viscosity, then activated via cyanylation with CDAP. Activated MenW is directly coupled to TT to form MenW-Tetanus Toxoid (MenW-TT).

Regarding the MenY-TT conjugate, MenY polysaccharide is first microfluidized to reduce molecular size and viscosity, then activated via cyanylation with CDAP. Activated MenY is directly coupled to TT to form MenY-Tetanus Toxoid (MenY-TT).

In one embodiment, the composition further includes a MenA_{AH}-TT conjugate having a mean TT/polysaccharide ratio 3; a MenC_{AH}-TT conjugate having a mean TT/polysaccharide ratio 3; a MenW-TT conjugate having a mean TT/polysaccharide ratio 1.5; and a MenY-TT conjugate having a mean TT/polysaccharide ratio 1.3. In a preferred embodiment, the composition includes a MenA_{AH}-TT conjugate having 5 mcg MenA polysaccharide and ˜15 mcg TT; a MenC_{AH}-TT conjugate having 5 mcg MenC polysaccharide and ˜15 mcg TT; a MenW-TT conjugate having 5 mcg MenW polysaccharide and ˜7.5 mcg TT; and a MenY-TT conjugate having 5 mcg MenY polysaccharide and ˜6.5 mcg TT. The composition may further include Tris-HCl, sucrose, and sodium chloride.

In another embodiment, the composition includes a MenA_{AH}-TT conjugate; MenC_{AH}-TT conjugate; MenW-TT conjugate; and MenY-TT conjugate, which includes MenA polysaccharide; MenC polysaccharide; MenW polysaccharide; and MenY polysaccharide and TT carrier protein. The composition may further include sucrose and Trometanol. For example, in one embodiment, the composition includes 10 μg/mL MenA polysaccharide; 10 μg/mL MenC polysaccharide; 10 μg/mL MenW polysaccharide; and 10 μg/mL MenY polysaccharide; 88 μg/mL TT carrier protein; 164 mM sucrose; and 1.6 mM Trometanol.

In one embodiment, the invention relates to use of a liquid immunogenic composition resulting from the lyophilized MenACWY-TT composition having been reconstituted with the liquid MenB bivalent rLP2086 composition. Reconstitution refers to restoring a dry lyophilized composition to a liquid form by the addition of a liquid diluent. In one preferred embodiment, the liquid MenB bivalent rLP2086 composition is not concomitantly administered, is not coadministered with, and is not simultaneously administered with the lyophilized MenACWY-TT composition, wherein the lyophilized MenACWY-TT composition has been reconstituted with a liquid composition that is not the liquid MenB bivalent rLP2086 composition. For example, in one preferred embodiment, the lyophilized MenACWY-TT composition is not reconstituted with an aqueous diluent consisting of sodium chloride and water and is not subsequently concomitantly administered, is not coadministered with, and is not simultaneously administered with the liquid MenB bivalent rLP2086 composition.

Rather, in a preferred embodiment, the lyophilized MenACWY-TT composition is administered with the MenB bivalent rLP2086 composition in one, i.e., a single, administration to the human. The resulting single administration (e.g., the MenABCWY composition) may result from the MenB bivalent rLP2086 composition, from a first container, being mixed with the lyophilized MenACWY-TT composition, from a second container. Alternatively, single administration of the MenABCWY composition may result from one (single) container that includes the MenB bivalent rLP2086 composition and the lyophilized MenACWY-TT composition. Delivery devices for vaccine or immunogenic compositions are known in the art. In one embodiment, the MenABCWY composition is administered concomitantly with any one of ibuprofen, paracetamol, and amoxicillin.

### First Polypeptide

The composition includes a first lipidated polypeptide and a second lipidated polypeptide. An exemplary first polypeptide in the composition may include a polypeptide having any one sequence selected from the sequences set forth in SEQ ID NO: 1, SEQ ID NO: 2, and SEQ ID NO: 6-74.

In one embodiment, the composition includes a first polypeptide having the amino acid sequence set forth in SEQ ID NO: 1. In one preferred embodiment, the composition includes about 60 μg of a first polypeptide including the amino acid sequence set forth in SEQ ID NO: 1, wherein the composition preferably has a total volume of 0.5 ml. In another embodiment, the composition includes about 120 μg/ml of a first polypeptide including the amino acid sequence set forth in SEQ ID NO: 1. The polypeptide is a modified factor H binding protein (fHBP) from N. meningitidis strain M98250771. A description of fHBP is disclosed in WO2012032489 and US patent publication US 2012/0093852, which are each incorporated by reference in their entirety. The polypeptide is N-terminally lipidated with three predominant fatty acids C16:0, C16:1, and C18:1 covalently linked at three positions of the polypeptide. The first polypeptide includes a total of 258 amino acids.

The embodiment wherein the first polypeptide includes SEQ ID NO: 1, the polypeptide includes two modifications introduced in the N-terminal region of the polypeptide, as compared to the corresponding wild-type sequence from N. meningitidis strain M98250771. A glycine in the second position is added as a consequence of introducing a cloning site. A second modification includes the deletion of four amino acids. Accordingly, in one embodiment, the first polypeptide includes a C-G-S-S sequence (SEQ ID NO: 3) at the N-terminus. See SEQ ID NO: 1, first four amino acid residues.

The N-terminal differences between the first polypeptide sequence and the wild-type Neisserial sequence is shown below. Accordingly, in one embodiment, the first polypeptide includes at least the first 2, 3, 4, 5, 6, 7, 8, 9, 10, 11, 12, 13, or more amino acid residues of the amino acid sequence set forth in SEQ ID NO: 1. Preferably, the first polypeptide includes at least the first 4, more preferably at least the first 6, and most preferably, at least the first 8 amino acid residues of SEQ ID NO: 1.

Comparison of Predicted N-Terminal Sequences of
Recombinant and Neisserial Subfamily A
LP2086 Polypeptide
rLP2086 M98250771
(SEQ ID NO: 4)
CGSS-----GGGGVAAD
Neisserial LP2086 M98250771
(SEQ ID NO: 5)
C-SSGS-GSGGGGVAAD
>A05
(SEQ ID NO: 1)
CGSSGGGGVAADIGTGLADALTAPLDHKDKGLKSLTLEDSISQNGTLTLS
AQGAEKTFKVGDKDNSLNTGKLKNDKISRFDFVQKIEVDGQTITLASGEF
QIYKQDHSAVVALQIEKINNPDKIDSLINQRSFLVSGLGGEHTAFNQLPS
GKAEYHGKAFSSDDAGGKLTYTIDFAAKQGHGKIEHLKTPEQNVELASAE
LKADEKSHAVILGDTRYGSEEKGTYHLALFGDRAQEIAGSATVKIREKVH
EIGIAGKQ

In one embodiment, the first polypeptide includes the amino acid sequence set forth in SEQ ID NO: 1. In one embodiment, the first polypeptide has a total of 258 amino acids. In one embodiment, the first polypeptide does not include an amino acid sequence having less than 100% sequence identity to SEQ ID NO: 1. In another embodiment, the first polypeptide consists of the amino acid sequence set forth in SEQ ID NO: 1. In another embodiment, the first polypeptide includes the amino acid sequence KDN. See for example, amino acid residues 73-75 of SEQ ID NO: 1. In another embodiment, the first polypeptide includes the amino acid sequence set forth in SEQ ID NO: 3 at the N-terminus of the polypeptide. In another embodiment, the first polypeptide includes the amino acid sequence set forth in SEQ ID NO: 4 at the N-terminus of the polypeptide.

In a preferred embodiment, the first polypeptide is readily expressed in a recombinant host cell using standard techniques known in the art. In another preferred embodiment, the first polypeptide includes a bactericidal epitope on the N- and/or C-domain of SEQ ID NO: 1. In one embodiment, the first polypeptide includes at least the first 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 1. Preferably, the first polypeptide includes at least the first 2, more preferably at least the first 4, and most preferably, at least the first 8 amino acid residues of SEQ ID NO: 1.

In another embodiment, the first polypeptide includes at least the last 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 1.

In one embodiment, the composition includes about 30 μg/ml of a first polypeptide including the amino acid sequence set forth in SEQ ID NO: 1. In one preferred embodiment, the composition includes about 60 μg of a first polypeptide including the amino acid sequence set forth in SEQ ID NO: 1. In one preferred embodiment, the composition includes about 60 μg of a first polypeptide including the amino acid sequence set forth in SEQ ID NO: 1, wherein the composition preferably has a total volume of 0.5 ml. In another embodiment, the composition includes about 120 μg/ml of a first polypeptide including the amino acid sequence set forth in SEQ ID NO: 1.

### Second Polypeptide

The composition includes a first lipidated polypeptide and a second lipidated polypeptide. An exemplary second polypeptide in the composition may include a polypeptide having any one sequence selected from the sequences set forth in SEQ ID NO: 1, SEQ ID NO: 2, and SEQ ID NO: 6-74.

In one embodiment, the composition includes a second polypeptide having the amino acid sequence set forth in SEQ ID NO: 2. In one preferred embodiment, the composition includes about 60 μg of a second polypeptide including the amino acid sequence set forth in SEQ ID NO: 2, wherein the composition preferably has a total volume of 0.5 ml. In another embodiment, the composition includes 120 μg/ml of a second polypeptide including the amino acid sequence set forth in SEQ ID NO: 2. The polypeptide is a factor H binding protein (fHBP) from N. meningitidis strain CDC1573. A description of fHBP is disclosed in WO2012032489 and US patent publication US 2012/0093852, which are each incorporated by reference in their entirety. The polypeptide is N-terminally lipidated with three predominant fatty acids C16:0, C16:1, and C18:1 covalently linked at three positions of the polypeptide. The second polypeptide includes a total of 261 amino acids. In one embodiment, the second polypeptide includes a C-G-S-S sequence (SEQ ID NO: 3) at the N-terminus. See the first four amino acid residues of SEQ ID NO: 2.

>B01
(SEQ ID NO: 2)
CGSSGGGGSGGGGVTADIGTGLADALTAPLDHKDKGLKSLTLEDSISQNG
TLTLSAQGAEKTYGNGDSLNTGKLKNDKVSRFDFIRQIEVDGQLITLESG
EFQVYKOSHSALTALQTEQEQDPEHSEKMVAKRRFRIGDIAGEHTSFDKL
PKDVMATYRGTAFGSDDAGGKLTYTIDFAAKQGHGKIEHLKSPELNVDLA
VAYIKPDEKHHAVISGSVLYNQDEKGSYSLGIFGEKAQEVAGSAEVETAN
GIHHIGLAAKQ

In one embodiment, the second polypeptide includes the amino acid sequence set forth in SEQ ID NO: 2. In one embodiment, the second polypeptide has a total of 261 amino acids. In one embodiment, the second polypeptide consists of the amino acid sequence set forth in SEQ ID NO: 2. In another embodiment, the second polypeptide does not further include a polypeptide having less than 100% sequence identity to SEQ ID NO: 2. In a preferred embodiment, the first polypeptide and the second polypeptide includes a C-G-S-S(SEQ ID NO: 3) sequence at the N-terminus of the respective polypeptide.

In a preferred embodiment, the second polypeptide is readily expressed in a recombinant host cell using standard techniques known in the art. In another preferred embodiment, the second polypeptide includes a bactericidal epitope on the N- and/or C-domain of SEQ ID NO: 2. In one embodiment, the second polypeptide includes at least the first 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 2. Preferably, the second polypeptide includes at least the first 2, more preferably at least the first 4, and most preferably, at least the first 8 amino acid residues of SEQ ID NO: 2.

In another embodiment, the second polypeptide includes at least the last 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues of the amino acid sequence set forth in SEQ ID NO: 2.

In one embodiment, the composition includes about 30 μg/ml of a polypeptide including the amino acid sequence set forth in SEQ ID NO: 2. In one preferred embodiment, the composition includes about 60 μg of a polypeptide including the amino acid sequence set forth in SEQ ID NO: 2. In one preferred embodiment, the composition includes about 60 μg of a second polypeptide including the amino acid sequence set forth in SEQ ID NO: 2, wherein the composition preferably has a total volume of 0.5 ml. In another embodiment, the composition includes 120 μg/ml of a second polypeptide including the amino acid sequence set forth in SEQ ID NO: 2.

### Saccharides

The term “saccharide” throughout this specification may indicate polysaccharide or oligosaccharide and includes both. Polysaccharides are isolated from bacteria or isolated from bacteria and sized to some degree by known methods and optionally by microfluidisation. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides.

Each N. meningitidis capsular saccharide may be conjugated to a carrier protein independently selected from the group consisting of TT, DT, CRM197, fragment C of TT and protein D. Although one or more N. meningitidis capsular saccharide may be conjugated to different carrier proteins from the others, in one embodiment they are all conjugated to the same carrier protein. For instance they may all be conjugated to the same carrier protein selected from the group consisting of TT, DT, CRM197, fragment C of TT and protein D. In this context CRM197 and DT may be considered to be the same carrier protein as they differ by only one amino acid. In a preferred embodiment all the N. meningitidis capsular saccharides present are conjugated to TT.

If the protein carrier is the same for 2 or more saccharides in the composition, the saccharide could be conjugated to the same molecule of the protein carrier (carrier molecules having 2 more different saccharides conjugated to it) [see for instance WO 04/083251; for example, a single carrier protein might be conjugated to MenA and MenC; MenA and MenW; MenA and MenY; MenC and MenW; MenC and MenY; Men W and MenY; MenA, MenC and MenW; MenA, MenC and MenY; MenA, MenW and MenY; MenC, MenW and MenY; MenA, MenC, MenW and MenY. Alternatively the saccharides may each be separately conjugated to different molecules of the protein carrier (each molecule of protein carrier only having one type of saccharide conjugated to it).

In one embodiment, at least 2 different saccharide conjugates are conjugated separately to the same type of carrier protein, wherein one or more saccharide(s) is/are conjugated to the carrier protein via a first type of chemical group on the protein carrier, and one or more saccharide(s) is/are conjugated to the carrier protein via a second (different) type of chemical group on the protein carrier.

In one embodiment the 2 conjugates involve the same saccharide linked to the same carrier, but by different conjugation chemistries. In an alternative embodiment 2 different saccharides are conjugated to different groups on the protein carrier.

By “conjugated separately to the same type of carrier protein” it is meant that the saccharides are conjugated to the same carrier individually (for example, MenA is conjugated to tetanus toxoid through an amine group on the tetanus toxoid and MenC is conjugated to tetanus toxoid through a carboxylic acid group on a different molecule of tetanus toxoid.)

The capsular saccharide(s) may be conjugated to the same carrier protein independently selected from the group consisting of TT, DT, CRM197, fragment C of TT and protein D. A more complete list of protein carriers that may be used in the conjugates of the invention is presented below. In this context CRM197 and DT may be considered to be the same carrier protein as they differ by only one amino acid. In an embodiment all the capsular saccharides present are conjugated to TT.

The saccharides may include any one of: N. meningitidis serogroup A capsular saccharide (MenA), N. meningitidis serogroup C capsular saccharide (MenC), N. meningitidis serogroup Y capsular saccharide (MenY), and N. meningitidis serogroup W capsular saccharide (MenW), or any combination thereof.

The first and second chemical groups present on the protein carrier are different from each other and are ideally natural chemical groups that may be readily used for conjugation purposes. They may be selected independently from the group consisting of: carboxyl groups, amino groups, sulphydryl groups, Hydroxyl groups, Imidazolyl groups, Guanidyl groups, and Indolyl groups. In one embodiment the first chemical group is carboxyl and the second is amino, or vice versa. These groups are explained in greater detail below.

In a specific embodiment the immunogenic composition comprises at least 2 different N. meningitidis capsular saccharides, wherein one or more is/are selected from a first group consisting of MenA and MenC which is/are conjugated to the carrier protein via the first type of chemical group on the protein carrier (for instance carboxyl), and one or more different saccharides is/are selected from a second group consisting of MenC, MenY and MenW which is/are conjugated to the carrier protein via the second type of chemical group on the protein carrier (for instance amino).

In a further embodiment the immunogenic composition of the invention comprises MenA conjugated via the first type of chemical group (for instance carboxyl), and MenC conjugated via the second type of chemical group (for instance amino).

In another embodiment the immunogenic composition comprises MenC conjugated via the first type of chemical group (for instance carboxyl), and MenY conjugated via the second type of chemical group (for instance amino).

In another embodiment the immunogenic composition comprises MenA conjugated via the first type of chemical group (for instance carboxyl), and MenC, MenY and MenW conjugated via the second type of chemical group (for instance amino).

In another embodiment the immunogenic composition comprises MenA and MenC conjugated via the first type of chemical group (for instance carboxyl), and MenY and MenW conjugated via the second type of chemical group (for instance amino).

The saccharides of the invention included in pharmaceutical (immunogenic) compositions of the invention are conjugated to a carrier protein such as tetanus toxoid (TT), tetanus toxoid fragment C, non-toxic mutants of tetanus toxin [note all such variants of TT are considered to be the same type of carrier protein for the purposes of this invention], diphtheria toxoid (DT), CRM197, other non-toxic mutants of diphtheria toxin [such as CRM176, CRM 197, CRM228, CRM 45 (Uchida et al J. Biol. Chem. 218; 3838-3844, 1973); CRM 9, CRM 45, CRM102, CRM 103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. Nos. 4,709,017 or 4,950,740; mutation of at least one or more residues Lys 516, Lys 526, Phe 530 and/or Lys 534 and other mutations disclosed in U.S. Pat. Nos. 5,917,017 or 6,455,673; or fragment disclosed in U.S. Pat. No. 5,843,711] (note all such variants of DT are considered to be the same type of carrier protein for the purposes of this invention), pneumococcal pneumolysin (Kuo et al (1995) Infect Immun 63; 2706-13), OMPC (meningococcal outer membrane protein—usually extracted from N. meningitidis serogroup B—EP0372501), synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al (2001) Eur J Immunol 31; 3816-3824) such as N19 protein (Baraldoi et al (2004) Infect Immun 72; 4884-7) pneumococcal surface protein PspA (WO 02/091998), iron uptake proteins (WO 01/72337), toxin A or B of C. difficile (WO 00/61761) or Protein D (EP594610 and WO 00/56360).

In an embodiment, the immunogenic composition of the invention uses the same type of carrier protein (independently) in at least two, three, four or each of the saccharides contained therein.

In an embodiment, the immunogenic composition of the invention comprises a N. meningitidis saccharide conjugated to a carrier protein selected from the group consisting of TT, DT, CRM197, fragment C of TT and protein D.

The immunogenic composition of the invention optionally comprises at least one meningococcal saccharide (for example MenA; MenC; MenW; MenY; MenA and MenC; MenA and MenW; MenA and MenY; MenC and Men W; Men C and MenY; Men W and MenY; MenA, MenC and MenW; MenA, MenC and MenY; MenA, MenW and MenY; MenC, MenW and MenY or MenA, MenC, MenW and MenY) conjugate having a ratio of Men saccharide to carrier protein of between 1:5 and 5:1, between 1:2 and 5:1, between 1:0.5 and 1:2.5 or between 1:1.25 and 1:2.5 (w/w). In one preferred embodiment, the composition includes MenA, MenC, MenW and MenY each conjugated to tetanus toxoid at ratios (toxoid to polysaccharide) of about 3, about 3, about 1.5 and about 1.3, respectively.

The ratio of saccharide to carrier protein (w/w) in a conjugate may be determined using the sterilized conjugate. The amount of protein is determined using a Lowry assay (for example Lowry et al (1951) J. Biol. Chem. 193, 265-275 or Peterson et al Analytical Biochemistry 100, 201-220 (1979)) and the amount of saccharide is determined using ICP-OES (inductively coupled plasma-optical emission spectroscopy) for MenA, DMAP assay for MenC and Resorcinol assay for MenW and MenY (Monsigny et al (1988) Anal. Biochem. 175, 525-530).

In an embodiment, the immunogenic composition of the invention comprises N. meningitidis saccharide conjugate(s) wherein the N. meningitidis saccharide(s) is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is ADH. Other linkers include B-propionamido (WO 00/10599), nitrophenyl-ethylamine (Geyer et al (1979) Med. Microbiol. Immunol. 165; 171-288), haloalkyl halides (U.S. Pat. No. 4,057,685), glycosidic linkages (U.S. Pat. Nos. 4,673,574, 4,808,700), hexane diamine and 6-aminocaproic acid (U.S. Pat. No. 4,459,286).

The saccharide conjugates present in the immunogenic compositions of the invention may be prepared by any known coupling technique. The conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a holoacetylated carrier protein (for example using iodoacetimide or N-succinimidyl bromoacetatebromoacetate). Optionally, the cyanate ester (optionally made by CDAP chemistry) is coupled with hexane diamine or ADH and the amino-derivatised saccharide is conjugated to the carrier protein using carbodiimide (e.g. EDAC or EDC) chemistry via a carboxyl group on the protein carrier. Such conjugates are described in PCT published application WO 93/15760 Uniformed Services University and WO 95/08348 and WO 96/29094.

Other suitable techniques use carbiinides, hydrazides, active esters, norborane, p-nitrobenzoic acid, N-hydroxysuccinimide, S-NHS, EDC, TSTU. Many are described in WO 98/42721. Conjugation may involve a carbonyl linker which may be formed by reaction of a free hydroxyl group of the saccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group’ reaction of the primary hydroxyl group with CDI to form a CDI carbamate intermediate and coupling the CDI carbamate intermediate with an amino group on a protein.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508.

A further method involves the coupling of a cyanogen bromide (or CDAP) activated saccharide derivatised with adipic acid hydrazide (ADH) to the protein carrier by Carbodiimide condensation (Chu C. et al Infect. Immunity, 1983 245 256), for example using EDAC.

In an embodiment, a hydroxyl group (optionally an activated hydroxyl group for example a hydroxyl group activated by a cyanate ester) on a saccharide is linked to an amino or carboxylic group on a protein either directly or indirectly (through a linker). Where a linker is present, a hydroxyl group on a saccharide is optionally linked to an amino group on a linker, for example by using CDAP conjugation. A further amino group in the linker for example ADH) may be conjugated to a carboxylic acid group on a protein, for example by using carbodiimide chemistry, for example by using EDAC. In an embodiment, N. meningitidis capsular saccharide(s) (or saccharide in general) is conjugated to the linker first before the linker is conjugated to the carrier protein. Alternatively the linker may be conjugated to the carrier before conjugation to the saccharide.

In general the following types of chemical groups on a protein carrier can be used for coupling/conjugation:

A) Carboxyl (for instance via aspartic acid or glutamic acid). In one embodiment this group is linked to amino groups on saccharides directly or to an amino group on a linker with carbodiimide chemistry e.g. with EDAC.
B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
C) Sulphydryl (for instance via cysteine). In one embodiment this group is linked to a bromo or chloro acetylated saccharide or linker with maleimide chemistry. In one embodiment this group is activated/modified with bis diazobenzidine.
D) Hydroxyl group (for instance via tyrosine). In one embodiment this group is activated/modified with bis diazobenzidine.
E) Imidazolyl group (for instance via histidine). In one embodiment this group is activated/modified with bis diazobenzidine.
F) Guanidyl group (for instance via arginine).
G) Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

### Direct Coupling Approaches:

Saccharide-OH+CNBr or CDAP----->cyanate ester+NH2-Prot---->conjugate
Saccharide-aldehyde+NH2-Prot---->Schiff base+NaCNBH3---->conjugate

### Saccharide-COOH+NH2-Prot+EDAC---->conjugate

### Saccharide-NH2+COOH-Prot+EDAC---->conjugate

### Indirect Coupling Via Spacer (Linker) Approaches:

Saccharide-OH+CNBr or CDAP--->cyanate ester+NH2----NH2---->saccharide----NH2+COOH-Prot+EDAC----->conjugate
Saccharide-OH+CNBr or CDAP---->cyanate ester+NH2-----SH----->saccharide----SH+SH-Prot (native Protein with an exposed cysteine or obtained after modification of amino groups of the protein by SPDP for instance)----->saccharide-S—S-Prot
Saccharide-OH+CNBr or CDAP--->cyanate ester+NH2----SH------->saccharide----SH+maleimide-Prot (modification of amino groups)---->conjugate
Saccharide-COOH+EDAC+NH2-----NH2--->saccharide------NH2+EDAC+COOH-Prot---->conjugate
Saccharide-COOH+EDAC+NH2----SH----->saccharide----SH+SH-Prot (native Protein with an exposed cysteine or obtained after modification of amino groups of the protein by SPDP for instance)----->saccharide-S—S-Prot
Saccharide-COOH+EDAC+NH2----SH----->saccharide----SH+maleimide-Prot (modification of amino groups)---->conjugate
Saccharide-Aldehyde+NH2------NH2---->saccharide---NH2+EDAC+COOH-Prot---->conjugate
Note: instead of EDAC above, any suitable carbodiimide may be used.

In summary, the types of protein carrier chemical group that may be generally used for coupling with a saccharide are amino groups (for instance on lysine residues), COOH groups (for instance on aspartic and glutamic acid residues) and SH groups (if accessible) (for instance on cysteine residues).

In an embodiment, at least one of the N. meningitidis capsular saccharides (or saccharide in general) is directly conjugated to a carrier protein; optionally Men W and/or MenY and/or MenC saccharide(s) is directly conjugated to a carrier protein. For example MenW; MenY; MenC; MenW and MenY; MenW and MenC; MenY and MenC; or MenW, MenY and MenC are directly linked to the carrier protein. Optionally, at least one of the N. meningitidis capsular saccharides is directly conjugated by CDAP. For example MenW; MenY; MenC; MenW and MenY; MenW and MenC; MenY and MenC; or MenW, MenY and MenC are directly linked to the carrier protein by CDAP (see WO 95/08348 and WO 96/29094). In an embodiment, all N. meningitidis capsular saccharides are conjugated to tetanus toxoid.

In an embodiment, the ratio of Men W and/or Y saccharide to carrier protein is between 1:0.5 and 1:2 (w/w) and/or the ratio of MenC saccharide to carrier protein is between 1:0.5 and 1:4 or 1:0.5 and 1:1.5 (w/w), especially where these saccharides are directly linked to the protein, optionally using CDAP.

In an embodiment, at least one of the N. meningitidis capsular saccharide(s) (or saccharide in general) is conjugated to the carrier protein via a linker, for instance a bifunctional linker. The linker is optionally heterobifunctional or homobifunctional, having for example a reactive amine group and a relative carboxylic acid group, 2 reactive amine groups or 2 reactive carboxylic acid groups. The linker has for example between 4 and 20, 4 and 12, 5 and 10 carbon atoms. A possible linker is ADH.

In an embodiment, MenA; MenC; or MenA and MenC is conjugated to a carrier protein (for example tetanus toxoid) via a linker.

In an embodiment, at least one N. meningitidis saccharide is conjugated to a carrier protein via a linker using CDAP and EDAC. For example, MenA; MenC; or MenA and MenC are conjugated to a protein via a linker (for example those with two hydrazino groups at its ends such as ADH) using CDAP and EDAC as described above. For example, CDAP is used to conjugate the saccharide to a linker and EDAC is used to conjugate the linker to a protein. Optionally the conjugation via a linker results in a ratio of saccharide to carrier protein of between 1:0.5 and 1:6; 1:1 and 1:5 or 1:2 and 1:4, for MenA; MenC; or MenA and MenC.

In an embodiment, the MenA capsular saccharide, where present is at least partially O-acetylated such that at least 50%, 60%, 70%, 80%, 90%, 95% or 98% of the repeat units are O-acetylated at at least one position. O-acetylation is for example present at least at the O-3 position of at least 50%, 60%, 70%, 80%, 90%, 95% or 98% of the repeat units.

In an embodiment, the MenC capsular saccharide, where present is at least partially O-acetylated such that at least 30%, 40%, 50%, 60%, 70%, 80%, 90%, 95% or 98% of (α2→9)-linked NeuNAc repeat units are O-acetylated at at least one or two positions. O-acetylation is for example present at the O-7 and/or O-8 position of at least 30%. 40%, 50%, 60%, 70%, 80%, 90%, 95% or 98% of the repeat units.

In an embodiment, the MenW capsular saccharide, where present is at least partially O-acetylated such that at least 30%, 40%, 50%, 60%, 70%, 80%, 90%, 95% or 98% of the repeat units are O-acetylated at at least one or two positions. O-acetylation is for example present at the O-7 and/or O-9 position of at least 30%. 40%, 50%, 60%, 70%, 80%, 90%, 95% or 98% of the repeat units.

In an embodiment, the MenY capsular saccharide, where present is at least partially O-acetylated such that at least 20%, 30%, 40%, 50%, 60%, 70%, 80%, 90%, 95% or 98% of the repeat units are O-acetylated at at least one or two positions. O-acetylation is present at the 7 and/or 9 position of at least 20%, 30%, 40%, 50%, 60%, 70%, 80%, 90%, 95% or 98% of the repeat units.

The percentage of O-acetylation refers to the percentage of the repeat units containing O-acetylation. This may be measured in the saccharide prior to conjugate and/or after conjugation.

In one embodiment of the invention the immunogenic composition, saccharide present, or each N. meningitidis capsular saccharide present, is conjugated to TT. In a further embodiment each N. meningitidis capsular saccharide is separately conjugated to a separate carrier protein. In a further embodiment each N. meningitidis capsular saccharide conjugate has a saccharide:carrier ratio of 1:5-5:1 or 1:1-1:4 (w/w). In a further embodiment at least one, two or three N. meningitidis capsular saccharide conjugate(s) is directly conjugated to a carrier protein. In a further embodiment Men W and/or MenY, MenW and/or MenC, MenY and/or MenC, or MenW and MenC and MenY are directly conjugated to a carrier protein. In a further embodiment at least one, two or three N. meningitidis saccharide conjugate(s) is directly conjugated by CDAP chemistry. In a further embodiment the ratio of Men W and/or Y saccharide to carrier protein is between 1:0.5 and 1:2 (w/w). In a further embodiment the ratio of MenC saccharide to carrier protein is between 1:0.5 and 1:2 (w/w). In a further embodiment at least one, two or three N. meningitidis capsular saccharide(s) are conjugated to the carrier protein via a linker (which may be bifunctional such as having two reactive amino groups (such as ADH) or two reactive carboxyl groups, or a reactive amino group at one end and a reactive carboxyl group at the other). The linker can have between 4 and 12 carbon atoms. In a further embodiment the or each N. meningitidis capsular saccharide(s) conjugated via a linker are conjugated to the linker with CDAP chemistry. In a further embodiment the carrier protein is conjugated to the linker using carbodiimide chemistry, for example using EDAC. In a further embodiment the or each N. meningitidis capsular saccharide is conjugated to the linker before the carrier protein is conjugated to the linker. In a further embodiment MenA is conjugated to a carrier protein via a linker (the ratio of MenA saccharide to carrier protein may be between 1:2 and 1:5 (w/w)). In a further embodiment MenC is conjugated to a carrier protein via a linker (the ratio of MenC saccharide to carrier protein may be between 1:2 and 1:5 (w/w)).

By using native or slightly sized polysaccharide conjugates, one or more of the following advantages may be realised: 1) a conjugate having high immungenicity which is filterable through a 0.2 micron filter; 2) immune memory may be enhanced (as in example three); 3) the alteration of the ratio of polysaccharide to protein in the conjugate such that the ratio of polysaccharide to protein (w/w) in the conjugate may be increased (this can result in a reduction of the carrier suppression effect); 4) immunogenic conjugates prone to hydrolysis (such as MenA conjugates) may be stabilised by the use of larger polysaccharides for conjugation. The use of larger polysaccharides can result in more cross-linking with the conjugate carrier and may lessen the liberation of free saccharide from the conjugate. The conjugate vaccines described in the prior art tend to depolymerise the polysaccharides prior to conjugation in order to improve conjugation. Meningococcal (or saccharide) conjugate vaccines retaining a larger size of saccharide can provide a good immune response against meningococcal disease.

The immunogenic composition of the invention may thus comprise one or more saccharide conjugates wherein the average size of each saccharide before conjugation is above 50 kDa, 75 kDa, 100 kDa, 110 kDa, 120 kDa or 130 kDa. In one embodiment the conjugate post conjugation should be readily filterable through a 0.2 micron filter such that a yield of more than 50, 60, 70, 80, 90 or 95% is obtained post filtration compared with the pre filtration sample.

In particular, the immunogenic composition of the invention comprises N. meningitidis capsular saccharides from at least one, two, three or four of serogroups A, C, W and Y conjugated to a carrier protein, wherein the average size (weight-average molecular weight; Mw) of at least one, two, three or four or each N. meningitidis saccharide is above 50 kDa, 60 kDa, 75 kDa, 100 kDa, 110 kDa, 120 kDa or 130 kDa.

In a preferred embodiment, the average Mw of the MenA_{AH}-TT conjugate is at least 250 kDa, 260 kDa, 270 kDa, 280 kDa, or 290 kDa, most preferably about 300 kDa, and at most 350 kDa or 330 kDa. In a preferred embodiment, the average Mw of the MenC_{AH}-TT conjugate is at least 150 kDa, 160 kDa, 170 kDa, 180 kDa, or 190 kDa, most preferably about 200 kDa, and at most 250 kDa or 230 kDa. In a preferred embodiment, the average Mw of the MenW-TT conjugate is at least 240, 250 kDa, 260 kDa, or 270 kDa, most preferably about 280 kDa, and at most 330 kDa or 310 kDa. In a preferred embodiment, the average Mw of the MenY-TT conjugate is at least 220 kDa, 230 kDa, 240 kDa, or 250 kDa, most preferably about 270 kDa, and at most 320 kDa or 300 kDa.

The immunogenic composition may comprise N. meningitidis capsular saccharides from at least one, two, three or four of serogroups A, C, W and Y conjugated to a carrier protein, wherein at least one, two, three or four or each N. meningitidis saccharide is either a native saccharide or is sized by a factor up to ×2, ×3, ×4, ×5, ×6, ×7, ×8, ×9 or ×10 relative to the weight average molecular weight of the native polysaccharide.

For the purposes of the invention, “native polysaccharide” refers to a saccharide that has not been subjected to a process, the purpose of which is to reduce the size of the saccharide. A polysaccharide can become slightly reduced in size during normal purification procedures. Such a saccharide is still native. Only if the polysaccharide has been subjected to sizing techniques would the polysaccharide not be considered native.

For the purposes of the invention, “sized by a factor up to ×2” means that the saccharide is subject to a process intended to reduce the size of the saccharide but to retain a size more than half the size of the native polysaccharide. ×3, ×4 etc. are to be interpreted in the same way i.e. the saccharide is subject to a process intended to reduce the size of the polysaccharide but to retain a size more than a third, a quarter etc. the size of the native polysaccharide.

In an aspect of the invention, the immunogenic composition comprises N. meningitidis capsular saccharides from at least one, two, three or four of serogroups A, C, W and Y conjugated to a carrier protein, wherein at least one, two, three or four or each N. meningitidis saccharide is native polysaccharide.

In an aspect of the invention, the immunogenic composition comprises N. meningitidis capsular saccharides from at least one, two, three or four of serogroups A, C, W and Y conjugated to a carrier protein, wherein at least one, two, three or four or each N. meningitidis saccharide is sized by a factor up to ×1.5, ×2, ×3, ×4, ×5, ×6, ×7, ×8, ×9 or ×10.

The immunogenic compositions of the invention optionally comprise conjugates of: N. meningitidis serogroup C capsular saccharide (MenC), serogroup A capsular saccharide (MenA), serogroup W capsular saccharide (MenW), serogroup Y capsular saccharide (MenY), serogroup C and Y capsular saccharides (MenCY), serogroup C and A capsular saccharides (MenAC), serogroup C and W capsular saccharides (MenCW), serogroup A and Y capsular saccharide (MenAY), serogroup A and W capsular saccharides (MenAW), serogroup W and Y capsular saccharides (Men WY), serogroup A, C and W capsular saccharide (MenACW), serogroup A, C and Y capsular saccharides (MenACY); serogroup A, W and Y capsular saccharides (MenAWY), serogroup C, W and Y capsular saccharides (MenCWY); or serogroup A, C, W and Y capsular saccharides (MenACWY). This is the definition of “one, two, three or four”, or “at least one of” of serogroups A, C, W and Y, or of each N. meningitidis saccharide where mentioned herein.

In an embodiment, the average size of at least one, two, three, four or each N. meningitidis saccharide is between 50 KDa and 1500 kDa, 50 kDa and 500 kDa, 50 kDa and 300 KDa, 101 kDa and 1500 kDa, 101 kDa and 500 kDa, 101 kDa and 300 kDa as determined by MALLS.

In an embodiment, the MenA saccharide, where present, has a molecular weight of 50-500 kDa, 50-100 kDa, 100-500 kDa, 55-90 KDa, 60-70 kDa or 70-80 kDa or 60-80 kDa.

In an embodiment, the MenC saccharide, where present, has a molecular weight of 100-200 kDa, 50-100 kDa, 100-150 kDa, 101-130 kDa, 150-210 kDa or 180-210 kDa.

In an embodiment the MenY saccharide, where present, has a molecular weight of 60-190 kDa, 70-180 kDa, 80-170 kDa, 90-160 kDa, 100-150 kDa or 110-140 kDa, 50-100 kDa, 100-140 kDa, 140-170 kDa or 150-160 kDa.

In an embodiment the MenW saccharide, where present, has a molecular weight of 60-190 kDa, 70-180 kDa, 80-170 kDa, 90-160 kDa, 100-150 kDa, 110-140 kDa, 50-100 kDa or 120-140 kDa.

The molecular weight or average molecular weight of a saccharide herein refers to the weight-average molecular weight (Mw) of the saccharide measured prior to conjugation and is measured by MALLS.

The MALLS technique is well known in the art and is typically carried out as described in example 2. For MALLS analysis of meningococcal saccharides, two columns (TSKG6000 and 5000PWxl) may be used in combination and the saccharides are eluted in water. Saccharides are detected using a light scattering detector (for instance Wyatt Dawn DSP equipped with a 10 mW argon laser at 488 nm) and an inferometric refractometer (for instance Wyatt Otilab DSP equipped with a P100 cell and a red filter at 498 nm).

In an embodiment the N. meningitidis saccharides are native polysaccharides or native polysaccharides which have reduced in size during a normal extraction process.

In an embodiment, the N. meningitidis saccharides are sized by mechanical cleavage, for instance by microfluidisation or sonication. Microfluidisation and sonication have the advantage of decreasing the size of the larger native polysaccharides sufficiently to provide a filterable conjugate (for example through a 0.2 micron filter). Sizing is by a factor of no more than ×20, ×10, ×8, ×6, ×5, ×4, ×3, ×2 or ×1.5.

In an embodiment, the immunogenic composition comprises N. meningitidis conjugates that are made from a mixture of native polysaccharides and saccharides that are sized by a factor of no more than ×20. For example, saccharides from MenC and/or MenA are native. For example, saccharides from MenY and/or MenW are sized by a factor of no more than ×20, ×10, ×8, ×6, ×5, ×4, ×3 or ×2. For example, an immunogenic composition contains a conjugate made from MenY and/or MenW and/or MenC and/or MenA which is sized by a factor of no more then ×10 and/or is microfluidised. For example, an immunogenic composition contains a conjugate made from native MenA and/or MenC and/or MenW and/or MenY. For example, an immunogenic composition comprises a conjugate made from native MenC. For example, an immunogenic composition comprises a conjugate made from native MenC and MenA which is sized by a factor of no more then ×10 and/or is microfluidised. For example, an immunogenic composition comprises a conjugate made from native MenC and MenY which is sized by a factor of no more then ×10 and/or is microfluidised.

In an embodiment, the polydispersity of the saccharide is 1-1.5, 1-1.3, 1-1.2, 1-1.1 or 1-1.05 and after conjugation to a carrier protein, the polydispersity of the conjugate is 1.0-2.5, 1.0-2.0. 1.0-1.5, 1.0-1.2, 1.5-2.5, 1.7-2.2 or 1.5-2.0. All polydispersity measurements are by MALLS.

Saccharides are optionally sized up to 1.5, 2, 4, 6, 8, 10, 12, 14, 16, 18 or 20 times from the size of the polysaccharide isolated from bacteria.

In one embodiment each N. meningitidis saccharide is either a native polysaccharide or is sized by a factor of no more than ×10. In a further embodiment each N. meningitidis capsular saccharide is a native polysaccharide. In a further embodiment at least one, two, three or four N. meningitidis capsular saccharide(s) is sized by microfluidization. In a further embodiment each N. meningitidis capsular saccharide is sized by a factor of no more than ×10. In a further embodiment the N. meningitidis conjugates are made from a mixture of native polysaccharides and saccharides that are sized by a factor of no more than ×10. In a further embodiment the capsular saccharide from serogroup Y is sized by a factor of no more than ×10. In a further embodiment capsular saccharides from serogroups A and C are native polysaccharides and saccharides from serogroups W and Y are sized by a factor of no more than ×10. In a further embodiment the average size of each N. meningitidis capular saccharide is between 50 kDa and 300 KDa or 50 kDa and 200 kDa. In a further embodiment the immunogenic composition comprises a MenA capsular saccharide having an average size of above 50 kDa, 75 kDa, 100 kDa or an average size of between 50-100 kDa or 55-90 KDa or 60-80 kDa. In a further embodiment the immunogenic composition comprises a MenC capsular saccharide having an average size of above 50 kDa, 75 kDa, 100 kDa or between 100-200 kDa, 100-150 kDa, 80-120 kDa, 90-110 kDa, 150-200 kDa, 120-240 kDa, 140-220 kDa, 160-200 kDa or 190-200 kDa. In a further embodiment the immunogenic composition comprises a MenY capsular saccharide, having an average size of above 50 kDa, 75 kDa, 100 kDa or between 60-190 kDa or 70-180 kDa or 80-170 kDa or 90-160 kDa or 100-150 kDa, 110-145 kDa or 120-140 kDa. In a further embodiment the immunogenic composition comprises a MenW capsular saccharide having an average size of above 50 kDa, 75 kDa, 100 kDa or between 60-190 kDa or 70-180 kDa or 80-170 kDa or 90-160 kDa or 100-150 kDa, 140-180 kDa, 150-170 kDa or 110-140 kDa.

In an embodiment of the invention, the saccharide dose of each of the at least two, three, four or each of the N. meningitidis saccharide conjugates is optionally the same, or approximately the same.

In an embodiment, the immunogenic composition of the invention is adjusted to or buffered at, or adjusted to between pH 7.0 and 8.0, pH 7.2 and 7.6 or around or exactly pH 7.4.

The immunogenic composition or vaccines of the invention are optionally lyophilised in the presence of a stabilising agent for example a polyol such as sucrose or trehalose.

For the N. meningitidis saccharide combinations discussed above, it may be advantageous not to use any aluminium salt adjuvant or any adjuvant at all.

The active agent can be present in varying concentrations in the pharmaceutical composition or vaccine of the invention. Typically, the minimum concentration of the substance is an amount necessary to achieve its intended use, while the maximum concentration is the maximum amount that will remain in solution or homogeneously suspended within the initial mixture. For instance, the minimum amount of a therapeutic agent is optionally one which will provide a single therapeutically effective dosage. For bioactive substances, the minimum concentration is an amount necessary for bioactivity upon reconstitution and the maximum concentration is at the point at which a homogeneous suspension cannot be maintained.

In another embodiment, the composition includes a conjugate of a Neisseria meningitidis serogroup X capsular polysaccharide and a carrier molecule. The structure of the group X capsular polysaccharide consists of N-acetylglucosamine-4-phosphate residues held together by α-(1-4) phosphodiester bonds without O-acetyl groups. The carrier molecule may be a diphtheria or tetanus toxoid, CRM 197 or protein D. In a preferred embodiment, as exemplified in the Examples, the composition does not include a conjugate of a N. meningitidis serogroup X capsular polysaccharide.

### Stability

The terms “stable” and “stability” refer the ability of an antigen to remain immunogenic over a period of time. Stability may be measured in potency over time. The terms “stable” and “stability” further refer to the physical, chemical, and conformational stability of the immunogenic composition. Instability of a protein composition may be caused by chemical degradation or aggregation of the protein molecules to form higher order polymers, by dissociation of the heterodimers into monomers, deglycosylation, modification of glycosylation, or any other structural modification that reduces at least one biological activity of the protein composition included in the present invention. Stability may be assessed by methods well-known in the art, including measurement of a sample's light scattering, apparent attenuation of light (absorbance, or optical density), size (e.g. by size exclusion chromatography), in vitro or in vivo biological activity and/or properties by differential scanning calorimetry (DSC). Other methods for assessing stability are known in the art and can also be used according to the present invention.

In some embodiments, an antigen in a stable formulation of the invention may maintain at least 50%, 60%, 70%, 75%, 80%, 85%, 90%, 95%, 96%, 97%, 98%, 99% or 100% potency, as compared to a reference standard, for at least 1 month, 2 months, 3 months, 4 months, 5 months, 6 months, 9 months, 12 months, 18 months, 24 months, 30 months, 36 months, 42 months, 48 months, 54 months, or 60 months. In some embodiments, an antigen in a stable formulation of the invention may maintain at least 50% potency, as compared to a reference standard, for at least 1 year, 2 years, 3 years, 4 years or 5 years. The terms “stable” and “stability” also refer to the ability of an antigen to maintain epitopes or immunoreactivity over a period of time. For example, an antigen in a stable formulation of the invention may maintain at least 50%, 60%, 70%, 75%, 80%, 85%, 90%, 95%, 96%, 97%, 98%, 99% or 100% of its epitopes or immunoreactivity, as compared to a reference standard, for at least 1 month, 2 months, 3 months, 4 months, 5 months, 6 months, 9 months, 12 months, 18 months, 24 months, 30 months, 36 months, 42 months, 48 months, 54 months, or 60 months. In some embodiments, stability is measured with respect to an environmental condition. Non-limiting examples of environmental conditions include light, temperature, freeze/thaw cycles, agitation, and pH. One of skill in the art would be able to determine the presence of antigenic epitopes or immunoreactivity using the methods disclosed herein or other methods known in the art. In some embodiments, the stability of an antigen is measured from the date of its formulation. In some embodiments, the stability of an antigen is measured from the date of a change in its storage conditions. Non-limiting examples of changes in storage conditions include changing from frozen to refrigerated, changing from frozen to room temperature, changing from refrigerated to room temperature, changing from refrigerated to frozen, changing from room temperature to frozen, changing from room temperature to refrigerated, changing from light to dark, or introduction of agitation.

In one embodiment, the terms “stable” and “stability” includes the ability of an antigen to be bound to aluminum. For example, a stable formulation of the invention includes at least 50%, 60%, 70%, 75%, 80%, 85%, 90%, 95%, 96%, 97%, 98%, 99% or 100% of a protein that is bound to aluminum (e.g., aluminum phosphate) in the formulation, as compared to a reference standard, for at least 1 hour, 6 hours, 12 hours, 18 hours, 24 hours, 1 month, 2 months, 3 months, 4 months, 5 months, 6 months, 9 months, 12 months, 18 months, 24 months, 30 months, 36 months, 42 months, 48 months, 54 months, or 60 months. See, for example Example 13. In a preferred embodiment, at least 90%, more preferably at least 95%, and most preferably at least 99% of the total Subfamily A rLP2086 polypeptide (e.g., a polypeptide that includes the amino acid sequence set forth in SEQ ID NO: 1) is bound to aluminum in the composition. In a preferred embodiment, at least 90%, more preferably at least 95%, and most preferably at least 99% of the total Subfamily B rLP2086 polypeptide (e.g., a polypeptide that includes the amino acid sequence set forth in SEQ ID NO: 2) is bound to aluminum in the composition.

Determination of Aluminum Binding. A composition comprising aluminum and at least one protein antigen was centrifuged such that the aluminum was pelleted. Centrifugation of aluminum absorbed proteins is known in the art. See e.g., Egan et al., Vaccine, Vol. 27(24): 3175-3180 (2009). Aluminum-bound protein was also pelleted, while non-aluminum-bound protein remained in the supernatant. Total protein in the supernatant and pellet were determined by Lowry Assay. The percentage bound protein was calculated by dividing the total protein in the supernatant by the total protein added to the composition and multiplying by 100%. Similarly, the percentage unbound protein was calculated by dividing the total protein in the supernatant by the total protein added to the composition and multiplying by 100%. For compositions comprising both Subfamily A and Subfamily B antigens, the individual Subfamily A and B protein concentrations in the supernatant were determined by ion-exchange chromatography. The separation and elution of Subfamily A and B proteins was carried out using a strong anion column and a high salt concentration eluent. Both Subfamily A and B proteins were detected and quantified using a fluorescence detector set at Excitation=280 run and Emission=310 run. Subfamily A and Subfamily B proteins elute at distinct retention times and were quantified using a standard curve generated against a rLP2086 protein reference material. The percentage unbound protein was calculated by dividing the total protein in the supernatant by the total protein added to the composition and multiplying by 100%. The percentage bound protein was calculated by subtracting the percentage unbound protein from 100%.

### Polysorbate-80

Polysorbate 80 (PS-80) is a non-ionic surfactant. Accelerated stability studies using an in vitro monoclonal antibody based potency assay demonstrated instability of the subfamily B protein at higher molar ratios of PS-80 to MenB rLP2086 protein in the final formulation. Further experiments with varying ratios of PS-80 have demonstrated that the optimal molar ratio of PS-80 to MenB rLP2086 protein is approximately 2.8±1.4 to retain potency.

The concentration of PS-80 in the composition is dependent on a molar ratio of PS-80 to the polypeptide. In one embodiment, the composition includes a 2.8±1.4 molar ratio of PS-80 to the first polypeptide and to the second polypeptide. In one embodiment, the composition includes a 2.8±1.1 molar ratio of PS-80 to the first polypeptide and to the second polypeptide. In one embodiment, the composition includes at least 1.9, 2.0, 2.1, 2.2, 2.3, 2.4, 2.5, 2.6, 2.7, 2.8, 2.9, 3.0, 3.1, 3.2, or 3.3 molar ratio of PS-80 to polypeptide. Preferably, the composition includes a 2.8 molar ratio of PS-80 to polypeptide.

The PS-80 to polypeptide molar ratio is determined by calculation from the measured concentration of PS-80 and the measured total polypeptide concentration, in which both values are expressed in moles. For example, PS-80 to Protein molar ratio is determined by calculation of the measured concentration of PS-80 (e.g., by reverse phase high pressure liquid chromatography (RP-HPLC)) to the measured total protein concentration (e.g., by ion exchange-high pressure liquid chromatography (IEX-HPLC)) in the final drug substance, where both values are expressed in moles.

A RP-HPLC is used to quantitate the concentration of Polysorbate 80 in vaccine formulations. The concentration of detergent is determined by saponification of the fatty acid moiety; Polysorbate 80 is converted to free oleic acid by alkaline hydrolysis at 40° C. The sample is separated by RP-HPLC using a C18 column and quantitated using a UV detector at a wavelength of 200 nm.

The first and the second polypeptides are resolved by anion-exchange HPLC. rLP2086(fHBP) Subfamily A and B proteins elute at distinct retention times and are quantitated using a standard curve generated against the respective rLP2086 protein reference material.

The term “molar ratio” and a description of an immunogenic composition including a fHBP and PS-80 is further disclosed in WO2012025873 and US patent publication US 2013/0171194, which are each incorporated by reference in their entirety.

The term “molar ratio” as used herein refers to the ratio of the number of moles of two different elements in a composition. In some embodiments, the molar ratio is the ratio of moles of detergent to moles of polypeptide. In some embodiments, the molar ratio is the ratio of moles of PS-80 to moles of protein. In one embodiment, based on the protein and Polysorbate 80 concentrations, the Molar Ratio may be calculated using the following equation:

$$Molar Ratio = \frac{\% PS - 80}{\text{mg/ml} protein} \times 216$$

In one embodiment, the composition includes about 0.0015, 0.0017, 0.0019, 0.0021, 0.0023, 0.0025, 0.0027, 0.0029, 0.0031, 0.0033, 0.0035, 0.0037, 0.0039, 0.0041, 0.0043, 0.0045, 0.0047, 0.0049, 0.0051 mg/mL PS-80. Preferably, the composition includes about 0.0035 mg/mL PS-80.

In another embodiment, the composition includes about 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, or 25 μg PS-80. In a preferred embodiment, the composition includes about 18 μg PS-80. In one embodiment, the composition does not comprise greater than 0.02 mg polysorbate-80.

In another embodiment, the composition includes a PS-80 concentration ranging from 0.0005% to 1%. For example, the PS-80 concentration in the composition may be at least 0.0005%, 0.005%, 0.01%, 0.02%, 0.03%, 0.04%, 0.05%, 0.06%, 0.07%, 0.08%, 0.09%, 0.10%, 0.2%, 0.3%, 0.4%, 0.5%, 0.6%, 0.7%, 0.8%, 0.9%, 1%, or 1.1% PS-80. In a preferred embodiment, the composition includes about 0.07% PS-80.

Any minimum value may be combined with any maximum value described herein to define a range.

### Aluminum

The composition preferably includes about 0.5 mg/ml aluminum phosphate. In one embodiment, the composition includes about 0.5 mg aluminum/ml as aluminum phosphate. AlPO₄ at 0.50 mg/ml is added as a stabilizer to provide enhanced manufacturability and stability. This concentration maintains binding (90% binding or better) of the subfamily A and B proteins to aluminum.

The process for producing an aluminum phosphate is described in US patent publication US 2009/0016946, which is incorporated by reference in its entirety.

In one embodiment, the composition does not further include a multivalent cation, other than aluminum. In one embodiment, the composition does not further include Al(OH)₃ or Al(SO₄)₃.

In one embodiment, the composition includes at least 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, or 250 μg aluminum. In one embodiment, the composition includes at most 500 μg, 490 μg, 480 μg, 470 μg, 460 μg, 450 μg, 440 μg, 430 μg, 420 μg, 410 μg, 400 μg, 390 μg, 380 μg, 370 μg, 360 μg, 350 μg, 340 μg, 330 μg, 320 μg, 310 μg, 300 μg, 290 μg, 280 μg, 270 μg, 260 μg, or 250 μg aluminum. Any minimum value may be combined with any maximum value described herein to define a range. In a most preferred embodiment, the composition includes 250 μg aluminum.

In one embodiment, the composition includes at least 0.005 mg/ml, 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.10 mg/ml, 0.2 mg/ml, 0.3 mg/ml, 0.4 mg/ml, or 0.5 mg/ml aluminum phosphate. In one embodiment, the composition includes at most 2.0 mg/ml, 1.9 mg/ml, 1.8 mg/ml, 1.7 mg/ml, 1.6 mg/ml, 1.5 mg/ml, 1.4 mg/ml, 1.3 mg/ml, 1.2 mg/ml, 1.1 mg/ml, 1.0 mg/ml, 0.9 mg/ml, 0.8 mg/ml, or 0.7 mg/ml PS-80. In a preferred embodiment, the composition includes about 0.07 mg/ml PS-80. Any minimum value may be combined with any maximum value described herein to define a range. In a preferred embodiment, the composition includes 0.5 mg/ml aluminum phosphate. In a most preferred embodiment, the composition includes 0.5 mg aluminum/ml as aluminum phosphate (AlPO₄). This concentration maintains binding (at least 90% binding or better) of the subfamily A and B proteins to aluminum.

in one embodiment, the percentage of total MenB rLP2086 polypeptides to the aluminum in the combined composition is at least 80%, 81%, 82%, 83%, 84%, 85%, 86%, 87%, 88%, 89%, 90%, 91%, 92%, 93%, 94%, 95%, 96%, 97%, 98%, 99%, or 100%. Preferably, the percentage of total MenB rLP2086 polypeptides to the aluminum in the combined composition is at least 90%, more preferably at least 95%, and most preferably at least 100%.

In another embodiment, the concentration of polypeptides bound to the aluminum in the immunogenic composition is not decreased after 24 hours, as compared to the concentration of polypeptides bound to the aluminum in the liquid composition prior to reconstituting the lyophilized composition. In another embodiment, the concentration of MenA_{AH}-TT conjugate in the immunogenic composition is not decreased after 24 hours, as compared to the concentration of the MenA_{AH}-TT conjugate in the lyophilized composition. In one embodiment, the concentration is decreased by at most 1%, 2%, 3%, 4%, 5%, 6%, 7%, 8%, 9%, 10%, 11%, 12%, 13%, 14%, 15%, 16%, 17%, 18%, 19%, or 20% after 24 hours, as compared to the respective concentration in the liquid composition prior to reconstitution.

In another embodiment, the concentration of MenC_{AH}-TT conjugate in the immunogenic composition is not decreased after 24 hours, as compared to the concentration of the MenC_{AH}-TT conjugate in the lyophilized composition. In another embodiment, the concentration of MenW-TT conjugate in the immunogenic composition is not decreased after 24 hours, as compared to the concentration of the MenW-TT conjugate in the lyophilized composition. In another embodiment, the concentration of MenY-TT conjugate in the immunogenic composition is not decreased after 24 hours, as compared to the concentration of the MenY-TT conjugate in the lyophilized composition. In one embodiment, the concentration is decreased by at most 1%, 2%, 3%, 4%, 5%, 6%, 7%, 8%, 9%, 10%, 11%, 12%, 13%, 14%, 15%, 16%, 17%, 18%, 19%, or 20% after 24 hours, as compared to the respective concentration in the lyophilized composition prior to reconstitution.

### Excipients

In one embodiment, the composition includes histidine. In one embodiment, the composition includes sodium chloride. The composition preferably includes about 10 mM histidine, and about 150 mM sodium chloride. In one embodiment, the composition includes 10 mM histidine and 150 mM sodium chloride.

In another embodiment, the composition includes about 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, or 850 μg of histidine. Preferably, the composition includes about 780 μg histidine. Any minimum value may be combined with any maximum value described herein to define a range.

In one embodiment, the composition includes a tris, phosphate, or succinate buffer. In a preferred embodiment, the composition does not include tris buffer. In a preferred, the composition does not include phosphate buffer. In one preferred embodiment, the composition does not include succinate buffer. In a preferred embodiment, the composition includes histidine buffer.

In one embodiment, the composition includes sodium chloride. Sodium chloride concentration in MenABCWY composition may vary between 160.5-161.1 mM.

In a preferred embodiment, the pH of the composition is between 6.0 and 7.0, most preferably pH 6.0. In one embodiment, the pH of the composition is at most 6.1. In one embodiment, the pH of the composition is between 5.5 and 7.5. In a preferred embodiment, the pH of the composition is between 5.8 and 7.0, most preferably pH 5.8 to pH 6.0. In one embodiment, the pH of the composition is at most 6.1. In one embodiment, the pH of the composition is 5.8.

### Kits

A further aspect of the invention is a kit for administering a dose of a composition for eliciting bactericidal antibodies against Neisseria meningitidis in a mammal.

In one aspect, the kit includes a first composition including a first polypeptide as described above and a second polypeptide as described above. In a preferred embodiment, the first polypeptide comprises the amino acid sequence set forth in SEQ ID NO: 1. In another preferred embodiment, the second polypeptide comprises the amino acid sequence set forth in SEQ ID NO: 2. The kit further includes a second composition including a MenA_{AH}-TT conjugate, a MenC_{AH}-TT conjugate, a MenW-TT conjugate, and a MenY-TT conjugate. In one embodiment, the kit includes at least two containers, wherein a first container includes the first composition, a second container includes the second composition.

In one embodiment, the kit includes a liquid first composition and a lyophilized second composition. Preferably, the kit includes a liquid MenB bivalent rLP2086 composition and a lyophilized MenACWY-TT composition.

The inventors surprisingly discovered that while a composition that includes a combination of the first composition and the second composition changes the molar ratio of polysorbate-80 in relation to the MenB rLP2086 polypeptides in the first composition, additional surfactant for the combined composition was surprisingly not necessary to maintain solubility and stability of the MenB rLP2086 polypeptides in the combined composition. Accordingly, in one embodiment, the kit does not comprise greater than 0.02 mg polysorbate-80.

In one embodiment of the invention, the kit does not further comprise any one of the following commercial immunogenic compositions: MENACTRA®, MENVEO®, ADACEL®, HAVRIX®, GARDASIL®, REPEVAX, or any combination thereof. For example, the kit preferably does not further include a meningococcal A, C, Y and W polysaccharide conjugate (MCV4) composition, wherein the carrier protein is diphtheria toxoid. In one embodiment, the kit does not further include a meningococcal A, C, Y and W polysaccharide conjugate (MCV4) composition, wherein the carrier protein is CRM₁₉₇. In one embodiment, the kit does not further comprise NIMENRIX vaccine, wherein NIMENRIX comprises a diluent consisting of sodium chloride and water.

### Bactericidal Activity

Immune response induced by administering the composition to a human is determined using a serum bactericidal assay using human complement (hSBA) against four N. meningitidis serogroup B (MenB) strains.

The high proportion of hSBA response to all test strains, especially strains expressing lipoprotein 2086 variants with sequences heterologous to the first polypeptide (SEQ ID NO: 1) suggests that the composition is a broadly protective vaccine and that two doses are sufficient to confer high seroprotection at least against N. meningitidis serogroup B subfamily A strains.

The high proportion of hSBA response to all test strains, especially strains expressing lipoprotein 2086 variants with sequences heterologous to both the first polypeptide (SEQ ID NO: 1) and the second polypeptide (SEQ ID NO: 2) suggests that the composition is a broadly protective vaccine and that at most three doses within about a 6 month period are sufficient to confer high seroprotection against N. meningitidis serogroup B strains expressing rLP2086 (FHBP) subfamily A and/or subfamily B.

### Subfamily A Strains

In one embodiment, the hSBA strain is an N. meningitidis strain that expresses LP2086 (fHBP) subfamily A protein. In one embodiment, the hSBA strain is an LP2086 (fHBP) subfamily A strain that expresses a lipoprotein 2086 variant that is heterologous to a N. meningitidis strain expressing A05. For example, in one embodiment, the hSBA strain is an LP2086 (fHBP) subfamily A strain that expresses a lipoprotein 2086 variant that is heterologous to strain M98250771.

In one embodiment, the hSBA strain is a N. meningitidis strain expressing fHBP A02. In one embodiment, the hSBA strain is a N. meningitidis strain expressing LP2086 (fHBP) A28. In one embodiment, the hSBA strain is a N. meningitidis strain expressing LP2086 (fHBP) A42. In a further embodiment, the hSBA strains are LP2086 (fHBP) A22 and LP2086 (fHBP) A63 strains. In another embodiment, the hSBA strain is a N. meningitidis strain expressing LP2086 A76.

In one embodiment, the hSBA strain is a N. meningitidis strain expressing fHBP A10. In one embodiment, the hSBA strain is a N. meningitidis strain expressing LP2086 (fHBP) A22. In one embodiment, the hSBA strain is a N. meningitidis strain expressing LP2086 (fHBP) A56. In another embodiment, the hSBA strain is a N. meningitidis strain expressing LP2086 A04. In one embodiment, the hSBA strain is a N. meningitidis strain expressing LP2086 A05. In one embodiment, the hSBA strain is a N. meningitidis strain expressing LP2086 A12. In one embodiment, the hSBA strain is a N. meningitidis strain expressing LP2086 A15. In one embodiment, the hSBA strain is a N. meningitidis strain expressing LP2086 A19. In one embodiment, the hSBA strain is a N. meningitidis strain expressing LP2086 A22. In one embodiment, the hSBA strain is a N. meningitidis strain expressing LP2086 A29. In one embodiment, the hSBA strain is a N. meningitidis strain expressing LP2086 A07. In one embodiment, the hSBA strain is a N. meningitidis strain expressing fHBP A62.

In one embodiment, the hSBA strain includes any one N. meningitidis strain expressing fHBP selected from the group consisting of: A02, A28, A42, A63, and A76, and any combination thereof. In one embodiment, the hSBA strain includes any one N. meningitidis strain expressing fHBP selected from the group consisting of: A02, A28, A42, A63, A76, A10, A22, A56, A04, A05, A12, A15, A19, A22, A29, and A07, and any combination thereof.

In one embodiment, the immune response is against N. meningitidis serogroup B A02 strain. In one embodiment, the immune response is against N. meningitidis serogroup B A28 strain. In one embodiment, the immune response is against N. meningitidis serogroup B A42 strain. A63 one embodiment, the immune response is against N. meningitidis serogroup B A76 strain.

In one embodiment, the immune response is against a N. meningitidis serogroup B strain selected from the group consisting of A02, A28, A42, A63, and A76. In one embodiment, the immune response is against a N. meningitidis serogroup B strain selected from the group consisting of A02, A28, A42, A63, A76, A10, A22, A56, A04, A05, A12, A15, A19, A22, A29, and A07, and any combination thereof.

In one embodiment, the immune response is bactericidal against a N. meningitidis serogroup B subfamily A strain that is heterologous to N. meningitidis strain M98250771. In one embodiment, the immune response is bactericidal against a N. meningitidis serogroup B subfamily A strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the first polypeptide (SEQ ID NO: 1). In another embodiment, the immune response is bactericidal against a N. meningitidis serogroup B subfamily A strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain M98250771. In a preferred embodiment, the immune response is bactericidal against a N. meningitidis serogroup B subfamily A strain that expresses a factor H binding protein including an amino acid sequence that has at least 80%, more preferably at least 84%, identity to a factor H binding protein expressed by N. meningitidis strain M98250771.

In another embodiment, the immune response is bactericidal against a N. meningitidis serogroup B subfamily A strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to the first polypeptide. In another embodiment, the immune response is bactericidal against a N. meningitidis serogroup B subfamily A strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain M98250771. In a preferred embodiment, the immune response is bactericidal against a N. meningitidis serogroup B subfamily A strain that expresses a factor H binding protein including an amino acid sequence that has at most 99%, more preferably at most 85%, identity to a factor H binding protein expressed by N. meningitidis strain M98250771. Any minimum value may be combined with any maximum value described herein to define a range.

### Subfamily B Strains

In one embodiment, the hSBA strain is an LP2086 (fHBP) subfamily B strain. In one embodiment, the hSBA strain is an LP2086 (fHBP) subfamily B strain that expresses a lipoprotein 2086 variant that is heterologous to a N. meningitidis strain expressing B01. For example, in one embodiment, the hSBA strain is an LP2086 (fHBP) subfamily B strain that expresses a lipoprotein 2086 variant that is heterologous to strain CDC1127. In a preferred embodiment, the hSBA strain is an LP2086 (fHBP) subfamily B strain that expresses a lipoprotein 2086 variant that is heterologous to strain CDC1573.

In one embodiment, the hSBA strain is a N. meningitidis strain expressing fHBP B05. In one embodiment, the hSBA strain is a N. meningitidis strain expressing LP2086 (fHBP) B07. In one embodiment, the hSBA strain is a N. meningitidis strain expressing LP2086 (fHBP) B08. In another embodiment, the hSBA strain is a N. meningitidis strain expressing LP2086 B13. In one embodiment, the hSBA strain is a N. meningitidis strain expressing LP2086 B52. In one embodiment, the hSBA strain is a N. meningitidis strain expressing LP2086 B107. In a further embodiment, the hSBA strain includes any one strain selected from the group consisting of B05, B07, B08, B13, B52 and B107. In a further embodiment, the hSBA strain includes any one strain selected from the group consisting of B05, B07, B08, B13, B52, B107, B01, B24, B44, B16, B03, B09, B15, and B153.

In one embodiment, the immune response is bactericidal against a N. meningitidis serogroup B subfamily B strain that is heterologous to a N. meningitidis strain expressing B01. In one embodiment, the immune response is against N. meningitidis serogroup B B05 strain. In one embodiment, the immune response is against N. meningitidis serogroup B B07 strain. In one embodiment, the immune response is against N. meningitidis serogroup B B08 strain. In one embodiment, the immune response is against N. meningitidis serogroup B B13 strain. In one embodiment, the immune response is against N. meningitidis serogroup B B52 strain. In one embodiment, the immune response is against N. meningitidis serogroup B B107 strain. In one embodiment, the immune response is against N. meningitidis serogroup B B24 strain. In one embodiment, the immune response is against N. meningitidis serogroup B B44 strain. In one embodiment, the immune response is against N. meningitidis serogroup B B16 strain. In one embodiment, the immune response is against N. meningitidis serogroup B B03 strain. In one embodiment, the immune response is against N. meningitidis serogroup B B09 strain. In one embodiment, the immune response is against N. meningitidis serogroup B B15 strain. In one embodiment, the immune response is against N. meningitidis serogroup B B153 strain. In one embodiment, the immune response is bactericidal against a N. meningitidis serogroup B subfamily B strain that is heterologous to N. meningitidis strain CDC1573.

In one embodiment, the immune response is against a N. meningitidis serogroup B strain selected from the group consisting of A02, A28, A42, A63, and A76. In one embodiment, the immune response is against a N. meningitidis serogroup B strain selected from the group consisting of B05, B07, B08, B13, B52, B107, B01, B24, B44, B16, B03, B09, B15, and B153, and any combination thereof.

In one embodiment, the immune response is bactericidal against a N. meningitidis serogroup B subfamily B strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the second polypeptide. In another embodiment, the immune response is bactericidal against a N. meningitidis serogroup B subfamily B strain that expresses a factor H binding protein including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain CDC1573. In a preferred embodiment, the immune response is bactericidal against a N. meningitidis serogroup B subfamily B strain that expresses a factor H binding protein including an amino acid sequence that has at least 80% identity, more preferably at least 87% identity, to a factor H binding protein expressed by N. meningitidis strain CDC1573. In another preferred embodiment, the immune response is bactericidal against a N. meningitidis serogroup B subfamily B strain that expresses a factor H binding protein including an amino acid sequence that has 100% identity to a factor H binding protein expressed by N. meningitidis strain CDC1573.

In another embodiment, the immune response is bactericidal against a N. meningitidis serogroup B subfamily B strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to the second polypeptide. In another embodiment, the immune response is bactericidal against a N. meningitidis serogroup B subfamily B strain that expresses a factor H binding protein including an amino acid sequence that has at most 81%, 82%, 83%, 84%, 85%, 86%, 87%, 88%, 89%, 90%, 91%, 92%, 93%, 94%, 95%, 96%, 97%, 98%, 99%, or 100% identity to a factor H binding protein expressed by N. meningitidis strain CDC1573. In a preferred embodiment, the immune response is bactericidal against a N. meningitidis serogroup B subfamily B strain that expresses a factor H binding protein including an amino acid sequence that has at most 99% identity, more preferably at least 88% identity, to a factor H binding protein expressed by N. meningitidis strain CDC1573. Any minimum value may be combined with any maximum value described herein to define a range.

In one embodiment, the hSBA strains include B05, B07, B08, B13, B52 and B107, and any combination thereof. In a further embodiment, the hSBA strains include B05, B07, B08, B13, B52 and B107, B24, B16, B44, B03, and B09, and any combination thereof. In one embodiment, the hSBA strains include A02, A28, A42, A63, A76, B05, B07, B08, B13, B52 and B107, and any combination thereof. In another embodiment, the hSBA strains further include A06, A07, A12, A15, A19, A29, B03, B09, B15, and B16, or any combination thereof. In another embodiment, the hSBA strains include A02, A28, A42, A63, A76, B05, B07, B08, B13, B52 and B107, A06, A07, A12, A15, A19, A29, B03, B09, B15, and B16, and any combination thereof.

### Subfamily A and Subfamily B Strains

In one embodiment, the method induces an immune response against a N. meningitidis serogroup B subfamily A strain and against a N. meningitidis serogroup B subfamily B strain. Preferably, the immune response is bactericidal against a N. meningitidis serogroup B subfamily A strain and against a N. meningitidis serogroup B subfamily B strain. In one embodiment, the method induces an immune response against a N. meningitidis serogroup B strain selected from the group consisting of A02, A28, A42, A63, A76, B05, B07, B08, B13, B52 and B107, and any combination thereof. In one embodiment, the method induces an immune response against a N. meningitidis serogroup B strain selected from the group consisting of A02, A28, A42, A63, A76, B05, B07, B08, B13, B52 and B107, A06, A07, A12, A15, A19, A29, B03, B09, B15, and B16, and any combination thereof.

In one embodiment, the method induces an immune response against a N. meningitidis serogroup B subfamily A strain and against a N. meningitidis serogroup B subfamily B strain. Preferably, the immune response is bactericidal against a N. meningitidis serogroup B subfamily A strain and against a N. meningitidis serogroup B subfamily B strain.

In one embodiment, the immune response against the N. meningitidis serogroup B subfamily A strain is greater than the immune response against the N. meningitidis serogroup B subfamily B strain. For example, in one embodiment, the immunogenic composition induces higher bactericidal titers against a N. meningitidis serogroup B subfamily A strain than against a N. meningitidis serogroup B subfamily B strain, when tested under identical conditions. In one embodiment, the higher bactericidal titers against a N. meningitidis serogroup B subfamily A strain occurs within 30 days after a second dose of the immunogenic composition against N. meningitidis. In one embodiment, the higher bactericidal titers against a N. meningitidis serogroup B subfamily A strain occur in the absence of a third dose of the immunogenic composition against N. meningitidis.

In another embodiment, the immune response against the N. meningitidis serogroup B subfamily B strain is greater than the immune response against the N. meningitidis serogroup B subfamily A strain. For example, in one embodiment, the immunogenic composition induces higher bactericidal titers against a N. meningitidis serogroup B subfamily B strain than against a N. meningitidis serogroup B subfamily A strain, when tested under identical conditions. In one embodiment, the higher bactericidal titers against a N. meningitidis serogroup B subfamily B strain occurs within 30 days after a second dose of the immunogenic composition against N. meningitidis. In one embodiment, the higher bactericidal titers against a N. meningitidis serogroup B subfamily B strain occur in the absence of a third dose of the immunogenic composition against N. meningitidis.

### Titers

In one embodiment, the composition induces an increase in bactericidal titer against N. meningitidis serogroup B in the human, as compared to the bactericidal titer against N. meningitidis serogroup B in the human prior to administration of a dose of the composition, when measured under identical conditions, e.g., in an hSBA. In one embodiment, the increase in bactericidal titer is compared to the bactericidal titer in the human before administration of the first dose of the composition, as compared to the bactericidal titer in the human prior to administration of the first dose of the composition, when measured under identical conditions, e.g., in an hSBA. In one embodiment, the increase in titer is observed after a second dose of the composition, as compared to the bactericidal titer in the human prior to administration of the second dose of the composition, when measured under identical conditions, e.g., in an hSBA. In another embodiment, the increase in bactericidal titer is observed after a third dose of the composition, as compared to the bactericidal titer in the human prior to administration of the third dose of the composition, when measured under identical conditions, e.g., in an hSBA.

In one embodiment, the composition induces a bactericidal titer against N. meningitidis serogroup B in the human after administration of a dose, wherein the bactericidal titer is at least greater than 1-fold higher than the bactericidal titer against N. meningitidis serogroup B in the human prior to administration of the dose, when measured under identical conditions, e.g., in an hSBA. For example, the bactericidal titer against N. meningitidis serogroup B may be at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 16-fold higher in the human after receiving a dose of the composition, as compared to the bactericidal titer against N. meningitidis serogroup B in the human prior to administration of the dose, when measured under identical conditions, e.g., in an hSBA.

In one embodiment, a “responder” refers to a human, wherein the composition induces a bactericidal titer against N. meningitidis serogroup B in the human after administration of a dose, wherein the bactericidal titer against N. meningitidis serogroup B is at least greater than 1-fold higher than the bactericidal titer against N. meningitidis serogroup B in the human prior to administration of the dose. In a preferred embodiment, the responder achieves at least a 4-fold rise in hSBA titer, as compared to a bactericidal titer in the human prior to administration of the dose. Such a responder may be referred to as having a protective titer. In some embodiments, a protective titer is one that is greater than 1:4.

In one embodiment, the hSBA titer is the reciprocal of the highest dilution of a serum sample that produces a measurable effect. For example, in one embodiment, the hSBA titer is the reciprocal of the highest 2-fold dilution of a test serum that results in at least a 50% reduction of MenB bacteria (50% bacterial survival) compared to the T30 CFU value (i.e., the number of bacteria surviving after incubation in assay wells containing all assay components except test serum; 100% bacterial survival).

In one embodiment, the composition induces a bactericidal titer against N. meningitidis serogroup B in the human after receiving the first dose that is at least 2-fold higher than the bactericidal titer against N. meningitidis serogroup B in the human prior to receiving the first dose (e.g., higher than the bactericidal titer in the human in the absence of the first dose), when measured under identical conditions in the hSBA. In one embodiment, the composition induces a bactericidal titer against N. meningitidis serogroup B in the human that is at least 4-fold higher than the bactericidal titer against N. meningitidis serogroup B in the human prior to receiving the first dose, when measured under identical conditions in a human serum bactericidal assay that utilizes human complement (hSBA). In one embodiment, the composition induces a bactericidal titer against N. meningitidis serogroup B in the human that is at least 8-fold higher than the bactericidal titer against N. meningitidis serogroup B in the human prior to receiving the first dose, when measured under identical conditions in a human serum bactericidal assay that utilizes human complement (hSBA).

In a preferred embodiment, the human serum complement is derived from a human having low intrinsic bactericidal activity for a given SBA test strain. Low intrinsic bactericidal activity refers to, for example, a bactericidal titer that is at least less than a 1:4 dilution against the given SBA test strain. In one embodiment, the human complement is derived from a human having an hSBA titer that is at least less than 1:4, such as a 1:2 dilution, against the given SBA test strain, wherein the composition was not administered to the human.

A human may exhibit an hSBA titer of less than 1:4 prior to administration of a composition, such as the bivalent rLP2086 composition, or a human may exhibit an hSBA titer of ≥1:4 prior to administration of the composition. Accordingly, in preferred embodiments and examples, administration of at least one dose of the composition to the human results in an hSBA titer that is at least greater than 1:4, such as, for example, an hSBA titer of ≥1:8, an hSBA titer of ≥1:16, and an hSBA titer of ≥1:32. The respective Examples described herein include assessments of the proportion of human subjects having an hSBA titer ≥1:8 and/or ≥1:16, wherein the bivalent rLP2086 composition was administered to the human. Such preferred assessments of hSBA titers greater than 1:4 show that the protection, i.e., the bactericidal immune response induced in the human, is associated with the composition.

In one embodiment, the human has an hSBA titer equal to or greater than the hSBA's lower limit of quantitation (LLOQ) after administration of the first dose of the composition. In another embodiment, the human has an hSBA titer equal to or greater than the hSBA's LLOQ after administration of the second dose of the composition. In another embodiment, the human has an hSBA titer equal to or greater than the hSBA's LLOQ after administration of the third dose of the composition.

### Methods and Administration

In one aspect, the invention relates to a method of inducing an immune response against N. meningitidis in a human. In another aspect, the invention relates to a method of vaccinating a human. In one embodiment, the method includes administering to the human at least one dose of the composition described above. In a preferred embodiment, the method includes administering to the human at most one dose of the composition described above. In another embodiment, the method includes administering to the human at least a first dose and a second dose of the composition described above.

In one embodiment, the second dose is administered at least 20, 30, 50, 60, 100, 120, 160, 170, or 180 days after the first dose, and at most 250, 210, 200, or 190 days after the first dose. Any minimum value may be combined with any maximum value described herein to define a range.

In another embodiment, the second dose is administered about 30 days after the first dose. In another embodiment, the second dose is administered about 60 days after the first dose, such as, for example, in a 0, 2 month immunization schedule. In another embodiment, the second dose is administered about 180 days after the first dose, such as, for example, in a 0, 6 month immunization schedule. In yet another embodiment, the second dose is administered about 120 days after the first dose, such as, for example, in a 2, 6 month immunization schedule.

In one embodiment, the method includes administering to the human two doses of the composition and at most two doses. In one embodiment, the two doses are administered within a period of about 6 months after the first dose. In one embodiment, the method does not include further administration of a booster to the human. A “booster” as used herein refers to an additional administration of the composition to the human. Administering to the human at most two doses of the composition may be advantageous. Such advantages include, for example, facilitating a human to comply with a complete administration schedule and facilitating cost-effectiveness of the schedule.

In one embodiment, the first dose and the second dose are administered to the human over a period of about 25, 30, 40, 50, 60, 70, 80, 90, 100, 110, 120, 130, 140, 150, 160, 170, 180, 190, or 200 days, and most 400, 390, 380, 370, 365, 350, 340, 330, 320, 310, 300, 290, 280, 270, 260, 250, 240, 230, 220, 210, or 200 days after the first dose. Any minimum value may be combined with any maximum value described herein to define a range. Preferably, the first and second doses will be administered at least 4 weeks apart e.g. ≥8 weeks apart, ≥2 months apart, ≥3 months apart, ≥6 months apart, etc.

In one embodiment, the first dose and the second dose are administered to the human over a period of about 30 days. In another embodiment, the first dose and the second dose are administered to the human over a period of about 60 days. In another embodiment, the first dose and the second dose are administered to the human over a period of about 180 days.

Conveniently, the first dose can be administered at substantially the same time as (e.g. during the same medical consultation or visit to a healthcare professional or within 24 hours of the first dose of the meningococcal vaccine) another vaccine e.g. at substantially the same time as a hepatitis B virus vaccine, a diphtheria vaccine, a tetanus vaccine, a pertussis vaccine (either cellular or, preferably, acellular), a Haemophilus influenzae type b vaccine, a Streptococcus pneumoniae vaccine, and/or a polio vaccine (preferably in inactivated poliovirus vaccine). Each of these optionally co-administered vaccines may be a monovalent vaccine or may be part of a combination vaccine (e.g. as part of a DTP vaccine).

Conveniently, the second dose can be administered at substantially the same time as (e.g. during the same medical consultation or visit to a healthcare professional or within 24 hours of the second dose of the meningococcal vaccine) another vaccine e.g. at substantially the same time as a hepatitis B virus vaccine, a diphtheria vaccine, a tetanus vaccine, a pertussis vaccine (either cellular or acellular), a Haemophilus influenzae type b vaccine, a Streptococcus pneumoniae vaccine, a polio vaccine (preferably in inactivated poliovirus vaccine), an influenza vaccine, a chickenpox vaccine, a measles vaccine, a mumps vaccine, and/or a rubella vaccine. Each of these optionally co-administered vaccines may be a monovalent vaccine or may be part of a combination vaccine (e.g. as part of an MMR vaccine).

Conveniently, the third dose can be administered at substantially the same time as (e.g. during the same medical consultation or visit to a healthcare professional or within 24 hours of the third dose of the meningococcal vaccine) another vaccine e.g. at substantially the same time as a hepatitis B virus vaccine, a diphtheria vaccine, a tetanus vaccine, a pertussis vaccine (either cellular or acellular), a Haemophilus influenzae type b vaccine, a Streptococcus pneumoniae vaccine, a polio vaccine (preferably in inactivated poliovirus vaccine), an influenza vaccine, a chickenpox vaccine, a measles vaccine, a mumps vaccine, and/or a rubella vaccine. Each of these optionally co-administered vaccines may be a monovalent vaccine or may be part of a combination vaccine (e.g. as part of an MMR vaccine).

### EXAMPLES

The following Examples illustrate embodiments of the invention. Unless noted otherwise herein, reference is made in the following Examples to a bivalent recombinant vaccine (rLP2086), which is a preferred exemplary embodiment of a composition including 60 μg of a first lipidated polypeptide including the amino acid sequence set forth in SEQ ID NO: 1 per 0.5 mL dose, 60 μg of a second lipidated polypeptide including the amino acid sequence set forth in SEQ ID NO: 2 per 0.5 mL dose, 2.8 molar ratio polysorbate-80 to the first polypeptide, 2.8 molar ratio polysorbate-80 to the second polypeptide, 0.5 mg Al³⁺/ml of the composition, 10 mM histidine, and 150 mM sodium chloride. More specifically, the bivalent recombinant rLP2086 vaccine (licensed as TRUMENBA) includes (a) 60 μg of a first lipidated polypeptide including the amino acid sequence set forth in SEQ ID NO: 1; (b) 60 μg of a second lipidated polypeptide including the amino acid sequence set forth in SEQ ID NO: 2; (c) 18 μg polysorbate-80; (d) 250 μg aluminum; (e) 780 μg histidine, and (0 4380 μg sodium chloride. Each dose was 0.5 mL.

### Example 1: Recombinant Antigens

Non-lipidated recombinant fHBP (rP2086) variants were expressed and purified. Mutations in the MN86-994-11 binding epitope were introduced by site directed mutagenesis. In this case, a His-tagged version of rP2086-601 cloned in plasmid vector pET30a was used as mutagenesis template to facilitate purification of recombinant mutants. A mutagenesis kit was used and mutagenic oligonucleotides used in the reaction were designed. The presence of intended mutations and the absence of secondary mutations were confirmed by DNA sequencing. Mutants expressed in E. coli strain BL21(DE3) were purified by Ni sepharose affinity chromatography and size exclusion chromatography. All CD and ITC experiments were done in 1×PBS, pH 7.4. Protein and antibody samples were thoroughly dialyzed against experimental buffer. Concentrations of rP2086-B01 (SEQ ID NO: 2) and MN86-994-11 were determined spectrophotometrically using extinction coefficients of 0.363 and 1.4 (mg/ml)⁻¹ cm⁻¹ at 280 nm, respectively. Light scattering was taking into account.

### Example 2: MEASURE Assay

A volume of 50 μL/well of bacteria fixed in 1% paraformaldehyde/PBS were plated into 96-well U-bottom polystyrene plates, centrifuged and washed once in 1% (w/v) BSA in 1×PBS. The mAb MN86-994-11-1 or mouse IgG (negative control) were added to the bacterial pellets, resuspended and incubated on ice for 30 minutes. After two washes, biotinylated goat anti-mouse IgG (subclasses 1+2a+2b+3) was added to the cell pellets, resuspended and incubated on ice for 30 minutes. The cells were washed twice and resuspended in streptavidin-PE and incubated on ice for 30 minutes. After an additional two washes, the cell pellets were resuspended in 1% PFA. 20,000 events per well were acquired on an Accuri C6 flow cytometer and analyzed using ACCURI CFLOW software. The mean fluorescent intensity (MFI) of the PE channel was determined for each sample after gating on bacterial cells in the logarithmic forward scatter versus side scatter dot plot. For fHBP expression to be considered above the LOD of the MEASURE assay, MFI values had to be above an arbitrary threshold of at least 100 and three times that of the control mouse IgG MFI in that assay. Serogroup B capsular expression was determined following the same staining procedure as previously described with the exception of the use of an anti-serogroup B mAb, followed by incubation with biotinylated goat anti-mouse IgM.

### Example 3: Serum Bactericidal Assay

hSBAs using human sera from young adults were performed. Human serum with no intrinsic detectable bactericidal activity in screening assays was used as the exogenous complement source. Subject matched pooled pre-immune sera were used to demonstrate that hSBA titers observed in the pooled post-immune sera was the result of vaccine-induced antibodies. Moreover, depletion experiments were performed to demonstrate the specificity of the antibodies for fHBP. Briefly, fHBP from the same subfamily competed with the binding of serum antibodies with antigen expressed on the surface of the bacteria and significantly reduced the hSBA titers, whereas irrelevant proteins and polysaccharides used as competitors did not (data not shown). In the study reported here, forty-five of the 109 NmB strains were tested with pre- and post-immune human sera from five subjects enrolled in the young adult clinical study 6108A1-500 (18-25 year old) and 64 NmB strains were tested with pre- and post-immune human sera from four of the same five subjects, due to insufficient serum available from the fifth subject. Strains were tested in hSBAs with the pooled human sera and up to 5 human serum complement lots. A strain tested in the hSBA was designated as killed if a 4-fold rise in hSBA titer was observed between the pre- and post-immune human serum samples for >50% of the assays that met system suitability criteria. This stringent approach was taken so that strains could be identified that could be used for clinical testing. In some instances, strains that could be killed by bivalent rLP2086 serum were scored negative as they could only be killed using specific complement sources. Appropriate system suitability was achieved if the ratio of the number of surviving bacteria after the bactericidal incubation in the absence of sera (T30) to the number of input bacteria (TO) was 50%. A strain was considered not susceptible (not killed) in the hSBA if a 4-fold rise in hSBA titer was not observed for >50% of the assays. As an example, if the hSBA titer of the pre-immune serum pool was <1:4 (or titer of 2) for a given NmB strain, then an hSBA titer of 1:8 with the post-immune serum pool would be required to achieve a 4-fold rise; if such a 4-fold rise was observed for >50% of the assays (e.g. 2 or 3 of 3 assays meeting system suitability criteria) a given NmB strain would be considered susceptible (killed) in the hSBA.

For the hSBA, human serum complement may be pooled from multiple normal healthy human adults or used from individual donors (i.e., not pooled).

### Example 4—Breadth of the Human Immune Response to TRUMENBA: Summary of fHBP Variants Expressed by MenB Strains that are Susceptible in the hSBA

Introduction & Aims: TRUMENBA (bivalent rLP2086), a vaccine for the prevention of Neisseria meningitis serogroup B (MenB) disease, includes two protein antigens, variants of meningococcal factor H binding protein (fHBP). fHBP exists as two subfamilies, A and B. Within each subfamily several hundred unique fHBP variants have been identified. Despite this sequence diversity, a vaccine containing one protein from each subfamily was demonstrated to induce broad coverage across MenB strains that represent the diversity of fHBP variants. Licensure was based on the ability of the vaccine to elicit antibodies that initiate complement-mediated killing of invasive MenB strains in a serum bactericidal assay using human complement (hSBA). Due to the endemic nature of meningococcal disease, it is not possible to predict which fHBP variants individuals may be exposed to. For this reason we have continued to explore the coverage conferred by TRUMENBA and present here additional evidence to illustrate the breadth of immune coverage.

Materials & Methods: MenB invasive strains (n=109) were selected to confirm TRUMENBA breadth of coverage. The strains encoded 22 and 16 unique subfamily A and subfamily B fHBP variants, respectively. The expression of fHBP at the bacterial surface was determined using the flow cytometric MEningococcal Antigen SURface Expression (MEASURE) assay. Exploratory hSBAs were performed using pre- and post-vaccination sera (subject-matched) from young adults. A strain was considered susceptible to TRUMENBA immune sera if a 4-fold rise in the hSBA titer was achieved between the pre- and post-vaccination serum samples.

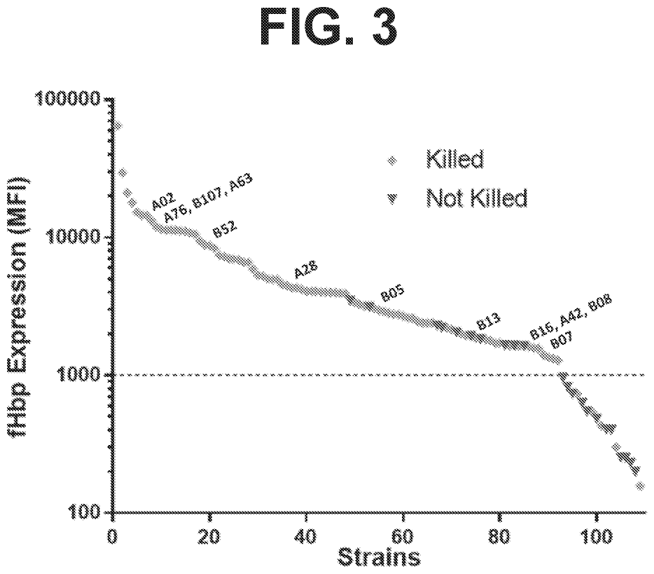

Results: Of the 109 strains, 87 (nearly 80%) were susceptible to TRUMENBA immune serum in hSBAs. This included strains expressing fHBP variants A02, A28, A42, A63, A76, B05, B07, B08, B13, B52 and B107, in addition to variants that had been reported previously. The majority of strains that could not be killed had fHBP expression levels that were below the level considered sufficient to initiate bactericidal killing in an hSBA. See FIG. 3, Table 1, Table 2, and Table 3.

TABLE 1

| | | fHBP | |
|---|---|---|---|
| | fHBP | Expression | Susceptible |
| Strain ID | Variant | (MFI) | in hSBA¹ |
| PMB3693 | A02 | 13157 | + |
| PMB876 | A28 | 4193 | + |
| PMB3106 | A42 | 1614 | + |
| PMB2871 | A63 | 10818 | + |
| PM B1606 | A76 | 11331 | + |
| PMB2627 | B05 | 2916 | + |
| PMB2219 | B07 | 1350 | + |
| PMB1610 | B08 | 1561 | + |
| PMB1486 | B13 | 1850 | + |
| PMB2466 | B52 | 8734 | + |
| PMB891 | B107 | 11125 | + |

TABLE 2
fHBP variants expressed by MenB strains that were killed
with TRUMENBA immune sera in hSBAs (% amino acid sequence
identity with A05 (SEQ ID NO: 1) and B01 (SEQ ID NO: 2))

| Subfamily A | Subfamily B |
|---|---|
| A02 (94.3) | B02 (92.0) |
| A04 (96.6) | B03 (90.8) |
| A05 (vaccine antigen) | B05 (87.7) |
| A06 (96.2) | B07 (87.3) |
| A07 (85.4) | B08 (87.7) |
| A12 (85.4) | B09 (88.1) |
| A15 (85.1) | B107 (89.7) |
| A17 (88.1) | B13 (86.9) |
| A19 (88.1) | B15 (86.5) |
| A22 (88.9) | B16 (86.2) |
| A26 (85.8) | B24 (86.2) |
| A28 (96.9) | B44 (91.6) |
| A42 (91.6) | B52 (91.9) |
| A56 (98.1) | |
| A63 (96.6) | |
| A76 (95.0) | |

TABLE 3
fHBP variants* expressed by strains at <1000 MFI
and were not killed with TRUMENBA immune sera in
hSBAs (% amino acid sequence identity with vaccine
antigens A05 (SEQ ID NO: 1) and B01 (SEQ ID NO: 2))

| Subfamily A | Subfamily B |
|---|---|
| A08 (85.9) | B10 (88.1) |
| A10 (85.4) | B91 (90.4) |
| A20 (87.7) | |
| A40 (85.1) | |
| A52 (96.6) | |

*These fHBP variants were only represented once in the strain collection.

Conclusion: The hSBA is recognized as the surrogate of efficacy for meningococcal vaccines. Assay complexity prevents demonstration of the bactericidal activity of TRUMENBA immune sera against MenB strains that express each of the hundreds of unique fHBP sequence variants. To illustrate the breadth of immune coverage conferred by TRUMENBA, we show that MenB strains expressing additional diverse fHBP variants can be killed in hSBAs despite being heterologous to the vaccine antigens.

### Example 5: Amino Acid Sequences

>fHBP_A02
(SEQ ID NO: 63)
CSSGGGGSGGGGVAADIGTGLADALTAPLDHKDKGLKSLTLEDSIPQNGT
LTLSAQGAEKTFKAGDKDNSLNTGKLKNDKISRFDFVQKIEVDGQTITLA
SGEFQIYKQDHSAVVALQIEKINNPDKIDSLINQRSFLVSGLGGEHTAFN
QLPGGKAEYHGKAFSSDDPNGRLHYSIDFTKKQGYGGIEHLKTPEQNVEL
ASAELKADEKSHAVILGDTRYGSEEKGTYHLALFGDRAQEIAGSATVKIG
EKVHEIGIAGKQ
>fHBP_A28
(SEQ ID NO: 64)
CSSGGGGSGGGGVAADIGTGLADALTAPLDHKDKGLKSLTLEDSIPQNGT
LTLSAQGAEKTFKAGDKDNSLNTGKLKNDKISRFDFVQKIEVDGQTITLA
SGEFQIYKQDHSAVVALQIEKINNPDKIDSLINQRSFLVSGLGGEHTAFN
QLPGGKAEYHGKAFSSDDAGGKLTYTIDFAAKQGHGKIEHLKTPEQNVEL
AAAELKADEKSHAVILGDTRYGSEEKGTYHLALFGDRAQEIAGSATVKIG
EKVHEISIAGKQ
>fHBP_A42
(SEQ ID NO: 65)
CSSGGGGVAADIGAGLADALTAPLDHKDKGLKSLTLEDSISQNGTLTLSA
QGAERTFKAGNKDNSLNTGKLKNDKISRFDFVQKIEVDGQTITLASGEFQ
IYKQNHSAVVALQIEKINNPDKIDSLINQRSFLVSSLGGEHTAFNQLPGG
KAEYHGKAFSSDDPNGRLHYSIDFTKKQGYGRIEHLKTPEQNVELAAAEL
KADEKSHAVILGDTRYGSEEKGTYHLALFGDRAQEIAGSATVKIGEKVHE
IGIAGKQ
>fHBP_A63
(SEQ ID NO: 66)
CSSGGGGSGGGGVAADIGTGLADALTAPLDHKDKGLKSLTLEDSIPQNGT
LTLSAQGAEKTFKAGDKDNSLNTGKLKNDKISRFDFVQKIEVDGQTITLA
SGEFQIYKQDHSAVVALQIEKINNPDKIDSLINQRSFLVSGLGGEHTAFN
QLPGGKAEYHGKAFSSDDAGGKLTYTIDFAAKQGHGKIEHLKTPEQNVEL
AAAELKADEKSHAVILGDTRYDSEEKGTYHLALFGDRAQEIAGSATVKIG
EKVHEISIAGKQ
>fHBP_A76
(SEQ ID NO: 67)
CSSGGGGSGGGGVAADIGAGLADALTAPLDHKDKGLKSLTLEDSIPQNGT
LTLSAQGAEKTFKAGDKDNSLNTGKLKNDKISRFDFVQKIEVDGQTITLA
SGEFQIYKQDHSAVVALQTEKVNNPDKTDSLINQRSFLVSGLGGEHTAFN
QLPVGKSEYHGKAFSSDDAGGKLTYTIDFAAKQGHGKIEHLKTPEQNVEL
AAAELKADEKSHAVILGDTRYGSEEKGTYHLALFGDRAQEIAGSATVKIG
EKVHEISIAGKQ
>fHBP_B05
(SEQ ID NO: 68)
CSSGGGGVAADIGAGLADALTAPLDHKDKGLQSLTLDQSVRKNEKLKLAA
QGAEKTYGNGDSLNTGKLKNDKVSRFDFIRQIEVDGQLITLESGEFQVYK
QSHSALTALQTEQVQDSEDSGKMVAKRQFRIGDIAGEHTSFDKLPKGGSA
TYRGTAFSSDDAGGKLTYTIDFAAKQGHGKIEHLKSPELNVELATAYIKP
DEKRHAVISGSVLYNQDEKGSYSLGIFGGQAQEVAGSAEVETANGIQHIG
LAAKQ
>fHBP_B07
(SEQ ID NO: 69)
CSSGGGGVAADIGAGLADALTAPLDHKDKGLQSLTLDQSVRKNEKLKLAA
QGAEKTYGNGDSLNTGKLKNDKVSRFDFIRQIEVDGKLITLESGEFQVYK
QSHSALTALQTEQVQDSEDSGKMVAKRQFRIGDIAGEHTSFDKLPKGGSA
TYRGTALGSDDAGGKLTYTIDFAAKQGHGKIEHLKSPELNVELATAYIKP
DEKRHAVISGSVLYNQDEKGSYSLGIFGGQAQEVAGSAEVETVNGIHHIG
LAAKQ
>fHBP_B08
(SEQ ID NO: 70)
CSSGGGGVAADIGAGLADALTAPLDHKDKSLQSLTLDQSVRKNEKLKLAA
QGAEKTYGNGDSLNTGKLKNDKVSRFDFIRQIEVDGKLITLESGEFQVYK
QSHSALTALQTEQVQDSEDSGKMVAKRQFRIGDIAGEHTSFDKLPKGGSA
TYRGTAFGSDDAGGKLTYTIDFAAKQGHGKIEHLKSPELNVELATAYIKP
DEKRHAVISGSVLYNQDEKGSYSLGIFGGQAQEVAGSAEVETANGIHHIG
LAAKQ
>fHBP_B13
(SEQ ID NO: 71)
CSSGGGGVAADIGAGLADALTAPLDHKDKGLQSLTLDQSVRKNEKLKLAA
QGAEKTYGNGDSLNTGKLKNDKVSRFDFIRQIEVDRQLITLESGEFQVYK
QSHSALTALQTEQVQDSEHSGKMVAKRQFRIGDIAGEHTSFDKLPEGGRA
TYRGTAFGSDDAGGKLIYTIDFAAKQGHGKIEHLKSPELNVDLAAADIKP
DEKHHAVISGSVLYNQAEKGSYSLGIFGGKAQEVAGSAEVKTVNGIRHIG
LAAKQ
>fHBP_B52
(SEQ ID NO: 72)
CSSGGGGVAADIGAGLADALTAPLDHKDKGLQSLTLDQSVRKNEKLKLAA
QGAEKTYGNGDSLNTGKLKNDKVSRFDFIRQIEVDGQLITLESGEFQVYK
QSHSALTALQTEQEQDLEHSGKMVAKRRFKIGDIAGEHTSFDKLPKDVMA
TYRGTAFGSDDAGGKLTYTIDFAAKQGHGKIEHLKSPELNVDLAVAYIKP
DEKHHAVISGSVLYNQDEKGSYSLGIFGEKAQEVAGSAEVKTANGIHHIG
LAAKQ
>fHBP_B107
(SEQ ID NO: 73)
CSSGGGGVAADIGAGLADALTAPLDHKDKGLKSLTLEDSISQNGTLTLSA
QGAERTFKAGDKDNSLNTGKLKNDKISRFDFIRQIEVDGQLITLESGEFQ
VYKQSHSALTALQTEQVQDSEHSGKMVAKRQFRIGDIVGEHTSFGKLPKD
VMATYRGTAFGSDDAGGKLTYTIDFAAKQGHGKIEHLKSPELNVDLAAAD
IKPDEKHHAVISGSVLYNQAEKGSYSLGIFGGQAQEVAGSAEVETANGIR
HIGLAAKQ
>A56
(SEQ ID NO: 74)
CSSGGGGVAADIGAGLADALTAPLDHKDKGLKSLTLEDSISQNGTLTLSA
QGAEKTFKVGDKDNSLNTGKLKNDKISRFDFVQKIEVDGQTITLASGEFQ
IYKQDHSAVVALQIEKINNPDKIDSLINQRSFLVSGLGGEHTAFNQLPSG
KAEYHGKAFSSDDAGGKLTYTIDFAAKQGHGKIEHLKTPEQNVELASAEL
KADEKSHAVILGDTRYGSEEKGTYHLALFGDRAQEIAGSATVKIREKVHE
IGIAGKQ

### Example 6: Selection of Diverse Strains to Assess Broad Coverage of the Bivalent FHbp Meningococcal B Vaccine

Although transmission of Neisseria meningitidis usually results in asymptomatic colonization of the upper respiratory tract, in some individuals, bacteremia and invasive meningococcal disease (IMD) occur. IMD commonly presents as meningitis and/or septicemia; pneumonia, septic arthritis, epiglottitis, and otitis media are less frequently observed. A high case fatality rate is associated with IMD (10%-15%), and approximately 20% of survivors have serious life-long sequelae such as limb amputation, hearing loss, and neurologic impairment.

Nearly all meningococcal disease worldwide is caused by 6 of the 12 characterized meningococcal serogroups (ie, A, B, C, W, X, and Y). Effective vaccines based on capsular polysaccharides have been developed for serogroups A, C, W, and Y. However, immunogenicity of the MenB polysaccharide is poor because of similarity to polysialic acid structures present on human neuronal cells. During recent years, meningococcal serogroup B (MenB) in particular has been associated with a large proportion of IMD in Europe, the United States, Canada, Australia, and New Zealand. Although vaccines based on outer membrane vesicles (OMVs) have been successfully used to control epidemics caused by a single MenB outbreak strain, the generated immune response is predominantly against the highly variable porin A protein (PorA). Therefore, effectiveness is generally limited to the target strain. Consequently, surface-exposed proteins capable of inducing protective bactericidal antibodies across diverse MenB strains have been sought for the development of a broadly effective MenB vaccine.

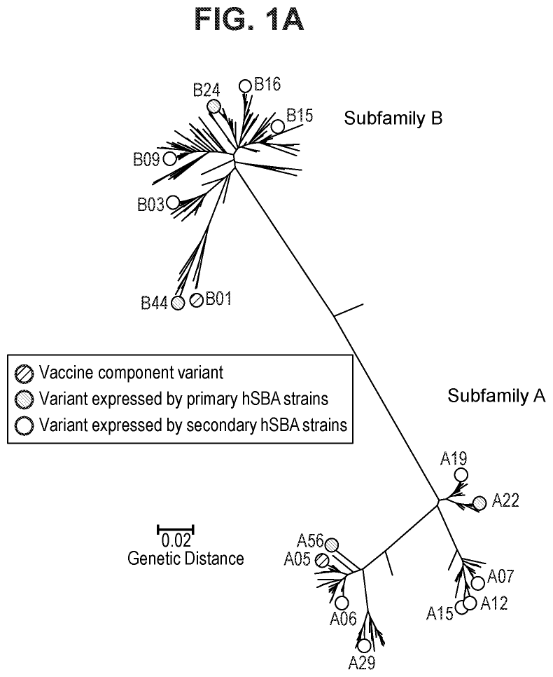

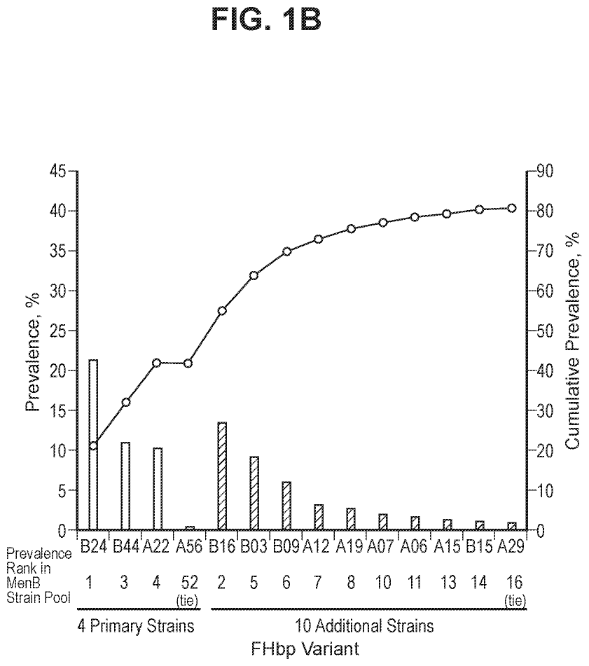

Factor H binding protein (FHbp; also known as LP2086 and GNA1870), a conserved surface-exposed lipoprotein expressed on nearly all strains of MenB, was identified as such a target. Based on amino acid sequence, FHbp variants segregate into 2 immunologically distinct subfamilies (termed subfamily A and subfamily B); each MenB strain expresses a single subfamily variant (see FIG. 1A).

MenB-FHbp (TRUMENBA®, bivalent rLP2086; Pfizer Inc, Philadelphia, Pa., USA) is a bivalent, recombinant protein MenB vaccine composed of equal amounts of 2 recombinant lipidated FHbp antigens, one from subfamily A (variant A05) and the other from subfamily B (variant B01). Importantly, it is predicted that this combination of FHbp variants is capable of providing protection against diverse MenB strains. MenB-FHbp has been approved for the prevention of IMD in several countries and regions, including the United States, Canada, Europe, and Australia. Another MenB vaccine, MenB-4C (Bexsero®, 4CMenB; GlaxoSmithKline Vaccines, Srl, Siena, Italy), also has a recombinant FHbp component (nonlipidated variant 1.1 from subfamily B) as well as 2 other recombinant protein antigens and an OMV. Thus, MenB-4C is different from MenB-FHbp, which contains two variants of a single antigen to afford broad coverage.

The serum bactericidal assay using human complement (hSBA) measures complement-dependent, antibody-mediated lysis of meningococcal bacteria. An hSBA titer is defined as the highest serum dilution killing ≥50% of assay bacteria; an hSBA titer ≥1:4 is the accepted correlate of protection against meningococcal disease, and hSBA response rates based on this correlate have been used as surrogates for meningococcal vaccine efficacy. The SBA response rate has been specifically correlated with natural protection for the serogroup C and A polysaccharide vaccines. Because serogroup-specific polysaccharides are not variable, a single strain from each serogroup was sufficient to infer broad vaccine coverage. MenB OMV vaccines are also efficacious and vaccine-elicited hSBA titers correlated with protection against the target strain causing the epidemic. Accurately predicting strain coverage of protein-based vaccines is more complex using hSBA than for vaccines targeting capsular polysaccharides, given that protein sequence diversity and variability in expression levels differ among the different meningococcal disease strains. For example, (PorA is the predominant target for serum bactericidal antibodies conferring protection after OMV vaccine immunization. PorA is a cell surface porin whose small cell surface exposed region has a high degree of sequence diversity. It has been estimated that protective immunity would need to be demonstrated with strains expressing 20 different PorA serosubtypes to protect against approximately 80% of sporadic MenB disease-causing strains in the United States. Historically, OMV vaccines have contained 1 PorA and have not demonstrated protection against strains with PorA sequences that are heterologous in amino acid sequence compared with the vaccine antigen. Therefore, selection of representative test strains to demonstrate that vaccine-elicited antibodies can be effective against a meningococcal disease strain is of paramount importance for protein-based vaccines.

Immune sera elicited by MenB-FHbp in preclinical and early clinical studies demonstrated broad bactericidal antibodies that could kill diverse MenB strains containing FHbp subfamily A and B variants heterologous to the vaccine FHbp variants A05 and B01. In an early assessment of the potential breadth of MenB-FHbp coverage, 100 MenB isolates with diverse FHbp variants, geographic origins, and genetic backgrounds were tested in hSBAs using MenB-FHbp immune rabbit serum. Of the 100 strains tested, 87 were killed in these hSBAs. Analysis of the 13 strains that were not killed suggested that the threshold FHbp surface expression level on a given MenB strain affected the hSBA response. A threshold FHbp surface expression level was subsequently determined, above which isolates were predictably killed in hSBA. Additional investigations of potential factors determining strain susceptibility found that killing was largely independent of FHbp sequence variant, multilocus sequence type, or PorA subtype.

To select strains with broad antigenic and epidemiologic diversity for clinical testing, over 1200 invasive MenB disease isolates were collected from laboratories and health agencies in the United States and Europe to represent the prevalence of MenB isolates that were contemporary at the time of collection; all strains contained the FHbp gene. An unbiased approach was used to select 4 antigenically and epidemiologically diverse representative test strains for use in MenB-FHbp immunogenicity studies. Selection criteria included expression of FHbp variants heterologous to the vaccine antigens and adequately reflecting the diversity of FHbp in MenB disease isolates, low to medium FHbp surface expression levels, and low baseline hSBA seropositivity rates. These 4 primary MenB test strains express FHbp variants from both FHbp subfamilies (strain [variant]: PMB2001 [A22], PMB80 [A56], PMB2707 [B24], and PMB2948 [B44]; see FIG. 1A).

To supplement immunogenicity data generated using the 4 primary MenB test strains and to demonstrate that immune responses against the 4 primary MenB test strains are predictive of immune responses against the diversity of FHbp variants expressed by MenB disease-causing isolates, hSBAs using 10 additional test strains were developed. The 10 additional test strains were selected to include prevalent FHbp variants found in MenB disease-causing strains in the United States and Europe. Here, we (i) describe the strategy and criteria used to select the 10 additional test strains, and (ii) present data demonstrating that the immune responses measured by hSBA using the 4 primary MenB strains are predictive of the responses obtained using 10 additional test strains, which further demonstrate and support the broad coverage of the immune response elicited by MenB-FHbp.

### Results

Sources and Selection Criteria for the Additional MenB Test Strains

Nine of the 10 additional MenB test strains were obtained from a collection of 1263 invasive disease-causing MenB strains (the MenB isolate collection). For the MenB isolate collection, US strains were from the Active Bacterial Core Surveillance sites (2000-2005), covering approximately 13% of the population. European isolates (2001-2006) were from the public health laboratories of Norway, France, Czech Republic and the Health Protection Agency in Manchester (which covers England, Wales, and Northern Ireland) and were collected systematically (every seventh or eighth isolate was included by order received at the country's reference laboratory) and represented approximately 13% of invasive MenB isolates during the period. The strains expressing FHbp variant A07 were obtained from an extension of the MenB isolate collection that included an additional 551 disease-causing MenB strains from Spain and Germany (n=1814). The extended MenB isolate collection was used as A07-expressing strains in the MenB isolate collection were not suitable because of the low surface expression of FHbp on these strains, high baseline seropositivity, and lack of readily available source of complement.

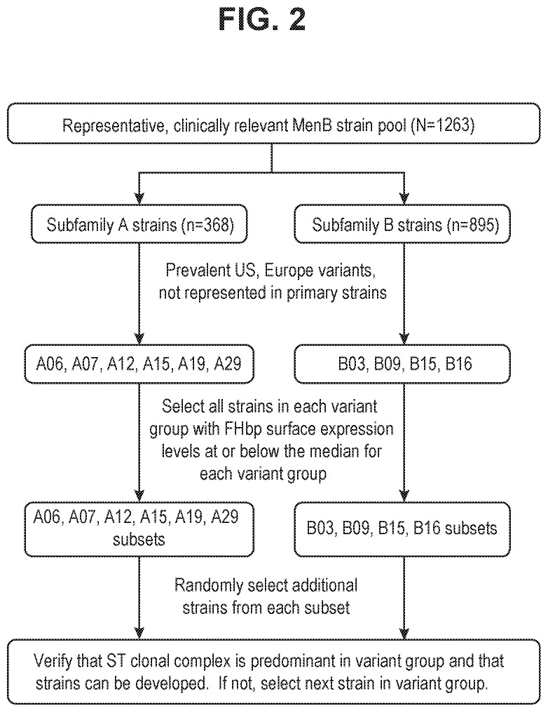

The criteria used to select the additional MenB test strains were (i) FHbp variant prevalence among MenB disease-causing strains in the United States and/or Europe, (ii) the FHbp variant needed to be different from those expressed by MenB primary test strains, (iii) in vitro FHbp expression levels at or below median levels for the respective FHbp variant group to ensure that the strain was representative of the variant group it belonged to, (iv) technical compatibility in the hSBA, and (v) being considered a predominant clonal complex for the variant group (if a predominant complex existed). Strains meeting these criteria also needed to be technically compatible in the hSBA, including adequate availability of suitable human complement lots (FIG. 2). Strains in each FHbp variant group with expression levels below the cutoff level (ie, at or below median levels for the respective FHbp variant group) were randomly selected, with the first strains within an FHbp variant group meeting the required genetic, phenotypic, and hSBA development criteria becoming the additional MenB test strains. An exception to this methodology was made for the strain expressing FHbp variant B03, which was selected in collaboration with and using guidance provided by the US FDA based on its previous use in a phase 2 study.

Characteristics of the Additional MenB Test Strains

The 10 additional selected MenB test strains express FHbp variants A06, A07, A12, A15, A19, A29, B03, B09, B15, and B16 which differ from the ones in the 4 primary test strains (A22, A56, B24, B44) and have different sequences compared to the vaccine antigens (Table 4). The specific variants expressed by the 4 primary test strains are present in 42.0% (530/1263) of disease-causing isolates in the MenB isolate collection, and the specific variants expressed by the 10 additional test strains are present in an additional, non-overlapping 38.8% (490/1263) of disease-causing isolates in the MenB isolate collection (FIG. 1B).

TABLE 4
Characteristics of the 4 Primary and 10 Additional MenB Test Strains

| | | | | | | |
|---|---|---|---|---|---|---|
| | | Percentage | | FHbp Variant | | |
| | | Identity to | Strain | Group MEASURE | | |
| | FHbp | Vaccine | MEASURE | MFI Median^{b} | Clonal | Country of |
| Strain | Variant | Component | MFI^{a} (±1 SD) | (±1 SD) | Complex | Isolation |

Primary Strains

| | | | | | | |
|---|---|---|---|---|---|---|
| PMB80 | A22 | 88.9 | 3127 | 2502 | CC41/44 | United States |
| | | | (2440, 4007) | (1952, 3207) | | |
| PMB2001 | A56 | 98.1 | 5002 | 5002^{c} | CC213 | France |
| | | | (3903, 6410) | | | |
| PMB2948 | B24 | 86.2 | 6967 | 8457 | CC32 | France |
| | | | (5436, 8929) | (6599, 10,839) | | |
| PMB2707 | B44 | 91.6 | 11,283 | 14,753 | CC269 | United Kingdom |
| | | | (8804, 14,461) | (11.511, 18,907) | | |

Additional Strains

| | | | | | | |
|---|---|---|---|---|---|---|
| PMB3010 | A06 | 96.2 | 3370 | 3088 | CC461 | United Kingdom |
| | | | (2629, 4319) | (2410, 3958) | | |
| PMB3040 | A07 | 85.4 | 1379 | 1100 | CC162 | Germany |
| | | | (1076, 1767) | (858, 1409) | | |
| PMB824 | A12 | 85.4 | 2540 | 2467 | CC35 | United States |
| | | | (1982, 3255) | (1925, 3161) | | |
| PMB1672 | A15 | 85.1 | 2995 | 2904 | CC103 | France |
| | | | (2337, 3838) | (2266, 3721) | | |
| PMB1989 | A19 | 88.1 | 1934 | 1759 | CC8 | United Kingdom |
| | | | (1509, 2479) | (1372, 2254) | | |
| PMB3175 | A29 | 93.1 | 3839 | 5994 | CC32 | United States |
| | | | (2995, 4920) | (4677, 7682) | | |
| PMB1256 | B03 | 90.8 | 3976 | 2935 | CC41/44 | United Kingdom |
| | | | (3102, 5096) | (2290, 3762) | | |
| PMB866 | B09 | 88.1 | 2089 | 2275 | CC269 | United Kingdom |
| | | | (1630, 2677) | (1775, 2916) | | |
| PMB431 | B15 | 86.5 | 3785 | 4822 | CC41/44 | United States |
| | | | (2953, 4851) | (3763, 6180) | | |
| PMB648 | B16 | 86.2 | 2347 | 1996 | CC41/44 | United Kingdom |
| | | | (1831, 3008) | (1557, 2558) | | |

FHbp = factor H binding protein; MenB = Neisseria meningitidis serogroup B; MFI = mean fluorescence intensity; SBA = serum bactericidal assay.
^{a}MFI ±1 SD from MEASURE assay.
^{b}Based on the MenB SBA isolate collection (n = 1263), except for variant group A07, which was calculated from the extended MenB SBA isolate collection (n = 1814). Strains in each FHbp variant group with expression levels at or below median levels for the respective FHbp variant group were randomly selected. The cutoff level adopted for each FHbp variant group was the observed median MFI plus 1 SD, using the precision estimate of 25.2% relative SD.
^{c}There is only one strain expressing A56; thus, no SD values are included.

Immunogenicity Analysis: Subjects with hSBA Titer ≥LLOQ for the 10 Additional Strains

The 4 primary strains were used to assess serological responses after 2 or 3 doses of MenB-FHbp in subjects participating in 2 pivotal phase 3 studies in adolescents and young adults. Serological responses to the 10 additional hSBA strains were assessed in a subgroup of the study subjects. The majority of subjects had hSBAs lower limit of quantitation (LLOQ; ie, hSBA titer equal to 1:8 or 1:16, depending on strain) 1 month after dose 2 and 1 month after dose 3 for each of the primary (64.0%-99.1% and 87.1%-99.5%, respectively) and the 10 additional MenB test strains (51.6%-100.0% and 71.3%-99.3%, respectively) (Table 5). For the primary and additional MenB test strains, a substantial increase from baseline in the proportion of subjects achieving an hSBA titer ≥LLOQ was observed among MenB-FHbp recipients (0, 2, 6 month schedule) after the second MenB-FHbp dose, with additional increases after the third dose.

TABLE 5
Subjects With hSBA Titers ≥ LLOQ (1:8 or
1:16) for Primary and Additional MenB Test Strains

| | % (95% CI) | | | | | |
|---|---|---|---|---|---|---|
| | [n] | | | | | |
| | Adolescents^{a} | | | Young Adults^{a} | | |
| | | 1 Month | 1 Month | | 1 Month | 1 Month |
| FHbp | | After | After | | After | After |
| Variant | Prevaccination | Dose 2 | Dose 3 | Prevaccination | Dose 2 | Dose 3 |

Primary strain

| | | | | | | |
|---|---|---|---|---|---|---|
| A22 | 33.2 (30.6, | 94.3 (92.9, | 97.8 (96.8, | 33.6 (31.3, | 84.7 (82.9, | 93.5 (92.2, |
| | 35.9) | 95.5) | 98.5) | 35.9) | 86.4) | 94.6) |
| | [1238] | [1263] | [1266] | [1704] | [1697] | [1714] |
| A56 | 27.5 (24.9, | 99.1 (98.4, | 99.5 (98.9, | 32.2 (29.9, | 97.4 (96.5, | 99.4 (98.9, |
| | 30.2) | 99.5) | 99.8) | 34.5) | 98.1) | 99.7) |
| | [1135] | [1222] | [1229] | [1657] | [1701] | [1708] |
| B24 | 6.4 (5.1, | 66.4 (63.6, | 87.1 (85.1, | 33.1 (30.9, | 86.5 (84.7, | 95.1 (93.9, |
| | 7.9) | 69.0) | 88.9) | 35.4) | 88.1) | 96.0) |
| | [1264] | [1216] | [1250] | [1696] | [1685] | [1702] |
| B44 | 3.6 (2.6, | 64.0 (61.3, | 89.3 (87.4, | 11.0 (9.6, | 68.3 (66.1, | 87.4 (85.8, |
| | 4.8) | 66.8) | 90.9) | 12.6) | 70.6) | 89.0) |
| | [1230] | [1204] | [1210] | [1716] | [1693] | [1703] |

Additional strain

| | | | | | | |
|---|---|---|---|---|---|---|
| A06 | 9.4 (6.2, | 84.0 (75.0, | 95.7 (92.6, | 16.0 (11.9, | 77.8 (67.8, | 92.0 (88.1, |
| | 13.5) | 90.8) | 97.8) | 20.9) | 85.9) | 94.9) |
| | [277] | [79] | [280] | [275] | [90] | [275] |
| A07 | 43.1 (37.1, | 93.8 (86.9, | 96.4 (93.5, | 55.8 (49.7, | 97.9 (92.6, | 95.7 (92.6, |
| | 49.3) | 97.7) | 98.3) | 61.8) | 99.7) | 97.7) |
| | [269] | [90] | [280] | [274] | [95] | [277] |
| A12 | 3.9 (2.0, | 67.4 (57.0, | 75.1 (69.6, | 5.0 (2.8, | 57.6 (46.9, | 71.3 (65.5, |
| | 6.9) | 76.6) | 80.1) | 8.3) | 67.9) | 76.5) |
| | [280] | [64] | [277] | [278] | [92] | [275] |
| A15 | 20.7 (16.1, | 65.6 (55.0, | 87.2 (82.6, | 37.3 (31.6, | 83.2 (74.1, | 91.8 (87.9, |
| | 26.1) | 75.1) | 91.0) | 43.2) | 90.1) | 94.7) |
| | [270] | [61] | [266] | [279] | [95] | [279] |
| A19 | 11.3 (7.8, | 84.5 (75.8, | 92.7 (89.0, | 28.8 (23.5, | 87.4 (79.0, | 95.8 (92.7, |
| | 15.7) | 91.1) | 95.5) | 34.5) | 93.3) | 97.8) |
| | [274] | [82] | [275] | [278] | [95] | [284] |
| A29 | 17.5 (13.1, | 100.0 (96.3, | 98.6 (96.4, | 31.1 (25.7, | 96.8 (91.0, | 99.3 (97.5, |
| | 22.5) | 100.0) | 99.6) | 36.9) | 99.3) | 99.9) |
| | [269] | [97] | [278] | [280] | [95] | [283] |
| B03 | 4.3 (2.2, | 61.1 (50.3, | 92.5 (88.7, | 11.2 (7.7, | 57.9 (47.3, | 86.4 (81.8, |
| | 7.4) | 71.2) | 95.3) | 15.5) | 68.0) | 90.3) |
| | [280] | [55] | [279] | [277] | [95] | [273] |
| B09 | 15.2 (11.2, | 76.3 (66.4, | 86.2 (81.6, | 23.5 (18.6, | 65.3 (54.8, | 77.0 (71.6, |
| | 19.9) | 84.5) | 90.1) | 28.9) | 74.7) | 81.9) |
| | [277] | [71] | [276] | [277] | [95] | [274] |
| B15 | 28.7 (23.5, | 96.8 (90.9, | 98.2 (95.9, | 43.8 (37.8, | 86.5 (78.0, | 96.7 (93.9, |
| | 34.5) | 99.3) | 99.4) | 49.9) | 92.6) | 98.5) |
| | [275] | [90] | [281] | [274] | [96] | [276] |
| B16 | 7.6 (4.8, | 61.6 (50.5, | 81.7 (76.6, | 21.9 (17.1, | 51.6 (41.1, | 78.0 (72.6, |
| | 11.4) | 71.9) | 86.0) | 27.3) | 62.0) | 82.8) |
| | [276] | [53] | [278] | [270] | [95] | [273] |

FHbp = factor H binding protein; hSBA = serum bactericidal assay using human complement; LLOQ = lower limit of quantitation; MenB = Neisseria meningitidis serogroup B.
Observed proportions of subjects were summarized with exact 2-sided 95% CIs using the Clopper-Pearson method.
LLOQ = 1:16 for A06, A12, A19, and A22; LLOQ = 1:8 for A07, A15, A29, A56, B03, B09, B15, B16, B24, and B44.
^{a}Evaluable immunogenicity population

Positive Predictive Values for the Primary and Additional Strains

The relationship between vaccine-induced hSBA responses for the primary MenB test strains and the 10 additional MenB test strains was assessed (

Table 6). Within an FHbp subfamily, positive predictive values (PPVs) were greater than 80% for most primary/additional strain pairs 1 month after dose 3. Thus, the immune responses measured in hSBAs using the primary test strains were highly predictive of immune responses for the additional strains within the same subfamily. The PPVs 1 month after dose 2 usually were slightly lower than those observed 1 month after dose 3 and ranged from 61.6% to 100% and 70.0% to 100% for subfamily A and B strain pairs, respectively, across studies. In summary, all PPVs showed high predictability for protective responses when comparing the primary and additional strain hSBA responses.

TABLE 6
Positive Predictive Value of Immune Response to Primary Strain for Immune
Response to Additional Strain following MenB-FHbp Vaccination

| | | % (95% CI)^{a} | | | |
|---|---|---|---|---|---|
| | | [n/N]^{b} | | | |
| FHbp Variant | | Adolescents | | Young Adults | |
| Primary | Additional Test | 1 Month After | 1 Month After | 1 Month After | 1 Month After |
| Test Strain | Strain | Dose 2 | Dose 3 | Dose 2 | Dose 3 |
| A22 | A06 | 89.7 (81.27, | 96.0 (92.90, | 87.5 (77.59, | 94.0 (90.26, |
| | | 95.16) | 97.97) | 94.12) | 96.59) |
| | | [78/87] | [262/273] | [63/72] | [234/249] |
| | A07 | 98.9 (93.83, | 96.3 (93.37, | 100.0 (95.20, | 99.2 (97.15, |
| | | 99.97) | 98.23) | 100.00) | 99.90) |
| | | [87/88] | [263/273] | [75/75] | [249/251] |
| | A12 | 72.7 (62.19, | 75.9 (70.37, | 67.6 (55.68, | 77.9 (72.24, |
| | | 81.68) | 80.90) | 78.00) | 82.91) |
| | | [64/88] | [205/270] | [50/74] | [194/249] |
| | A15 | 70.9 (60.14, | 89.7 (85.31, | 92.4 (84.20, | 93.9 (90.27, |
| | | 80.22) | 93.18) | 97.16) | 96.47) |
| | | [61/86] | [227/253] | [73/79] | [246/262] |
| | A19 | 87.8 (79.18, | 95.4 (92.11, | 97.5 (91,15, | 98.9 (96,76, |
| | | 93.74) | 97.60) | 99.69) | 99.77) |
| | | [79/90] | [249/261] | [77/79] | [265/268] |
| | A29 | 100.0 (95.98, | 99.6 (97.91, | 98.7 (93.15, | 100.0 (98.62, |
| | | 100.00) | 99.99) | 99.97) | 100.00) |
| | | [90/90] | [263/264] | [78/79] | [266/266] |
| A56 | A06 | 84.3 (75.02, | 96.3 (93.29, | 83.3 (73.62, | 93.0 (89.23, |
| | | 91.12) | 98.21) | 90.58) | 95.71) |
| | | [75/89] | [260/270] | [70/84] | [251/270] |
| | A07 | 94.4 (87.37, | 97.0 (94.22, | 98.9 (93.90, | 96.0 (92.88, |
| | | 98.15) | 98.71) | 99.97) | 97.96) |
| | | [84/89] | [261/269] | [88/89] | [261/272] |
| | A12 | 68.2 (57.39, | 75.6 (69.94, | 61.6 (50.51, | 72.2 (66.47, |
| | | 77.71) | 80.61) | 71.92) | 77.48) |
| | | [60/88] | [201/266] | [53/86] | 1195/270] |
| | A15 | 64.4 (53.38, | 89.2 (84.68, | 84.6 (75.54, | 92.0 (88.10, |
| | | 74.35) | 92.76) | 91.33) | 94.90) |
| | | [56/87] | [223/250] | [77/91] | [252/2741 |
| | A19 | 83.5 (74.27, | 93.8 (90.12, | 90.1 (82.05, | 96.4 (93.48, |
| | | 90.47) | 96.41) | 95.38) | 98.26) |
| | | [76/91] | [242/258] | [82/91] | 1268/278] |
| | A29 | 100.0 (96.03, | 98.9 (96.68, | 97.8 (92.29, | 99.6 (98.01, |
| | | 100.00) | 99.76) | 99.73) | 99.99) |
| | | [91/91] | [258/261] | [89/91] | [276/277] |
| B24 | B03 | 80.3 (68.16, | 97.1 (94.16, | 75.7 (63.99, | 89.9 (85.53, |
| | | 89.40) | 98.83) | 85.17) | 93.28) |
| | | [49/61] | [236/243] | [53/70] | [231/257] |
| | B09 | 88.7 (78.11, | 92.1 (87.96, | 82.9 (71.97, | 80.5 (75.17, |
| | | 95.34) | 95.19) | 90.82) | 85.20) |
| | | [55/62] | [222/241] | [58/70] | [207/257] |
| | B15 | 100.0 (94.22, | 99.6 (97.75, | 100.0 (94.87, | 98.8 (96.67, |
| | | 100.00) | 99.99) | 100.00) | 99.76) |
| | | [62/62] | [244/245] | [70/70] | [257/260] |
| | B16 | 82.1 (69.60, | 86.4 (81.46, | 70.0 (57.87, | 81.3 (76.01, |
| | | 91.09) | 90.46) | 80.38) | 85.90) |
| | | [46/56] | [210/243] | [49/70] | [209/257] |
| B44 | B03 | 78.9 (66.11, | 96.6 (93.40, | 88.9 (77.37, | 95.8 (92.38, |
| | | 88.62) | 98.52) | 95.81) | 97.96) |
| | | [45/57] | [227/235] | [48/54] | [227/237] |
| | B09 | 88.3 (77.43, | 90.1 (85.50, | 96.4 (87.47, | 85.9 (80.77, |
| | | 95.18) | 93.61) | 99.56) | 90.09) |
| | | [53/60] | [209/232] | [53/55] | [201/234] |
| | B15 | 100.0 (94.04, | 99.2 (96.99, | 100.0 (93.51, | 98.3 (95,74, |
| | | 100.00) | 99.90) | 100.00) | 99.54) |
| | | [60/60] | [235/237] | [55/55] | [233/237] |
| | B16 | 84.9 (72.41, | 85.5 (80.37, | 79.6 (66.47, | 83.8 (78.40, |
| | | 93.25) | 89.77) | 89.37) | 88.24) |
| | | [45/53] | [201/235] | [43/54] | [196/234] |

hSBA = serum bactericidal assay using human complement; LLOQ = lower limit of quantitation; MenB = Neisseria meningitidis serogroup B.
LLOQ = 1:8 for strains expressing variants A07, A15, A29, A56, B03, B09, B15, B16, B24, and B44; LLOQ = 1:16 for strains expressing variants A06, A12, A19, and A22.
^{a}Exact 2-sided CI based on the observed proportion of subjects using the Clopper-Pearson method.
^{b}N = number of subjects with valid and determinate assay results for both the primary and additional strains with observed hSBA titer ≥ LLOQ for the primary strain at 1 month after vaccination 2 and at 1 month after vaccination 3; n = number of subjects with observed hSBA titer ≥ LLOQ for the given additional strain at 1 month after vaccination 2 and at 1 month after vaccination 3.

Discussion

A critical component of the clinical evaluation of the MenB-FHbp vaccine to determine the breadth of protection was the development of hSBAs using test strains with surface protein antigens whose sequence and expression variability are representative of the diversity of MenB disease-causing strains that were contemporary at the time of collection. As described in phase 3 studies in adolescents and young adults, hSBA response data for the 4 primary MenB test strains, all of which express FHbp variants heterologous to the vaccine antigens, strongly suggest that the bivalent MenB-FHbp vaccine provides broad coverage across diverse, disease-causing meningococcal strains. The 10 additional MenB test strains described here provide supportive immunologic data for MenB-FHbp and further confirm the validity of the use of the 4 primary test strains to measure the immune response to MenB-FHbp. As the responses obtained for the 4 primary test strains are predictive of the responses obtained for the additional 10 test strains, the immunological responses obtained by assessing the primary strains in hSBAs are representative of the diversity of strains causing invasive MenB disease.

For the hypothesis test-driven immunogenicity evaluations in licensure studies for MenB-FHbp, an unbiased approach was used to select the 4 primary MenB test strains from panels of disease-causing MenB collected in the United States and Europe. A similar method was used to select the 10 additional MenB hSBA test strains, taking into consideration specific selection criteria to ensure that test strains were representative of the antigenic diversity of MenB isolates. Collectively, the 14 MenB test strains represent the majority of the prevalent meningococcal FHbp, with FHbp variants corresponding to approximately 80% of circulating invasive disease-causing isolates in the United States and Europe.

Positive predictive value analyses were used to determine the association of immune responses, measured by hSBA, among primary and additional test strains expressing FHbps within the same subfamily. All of the PPV analyses showed the high predictability of the protective responses against the primary strain for the protective responses observed against the additional strains. These PPV analyses indicate that the responses observed against the 4 primary MenB test strains are representative of responses to other disease-causing MenB strains that express additional sequence-diverse FHbp variants different from the vaccine antigen variants.

The MenB-FHbp-elicited responses measured by hSBA to the 4 primary and 10 additional MenB test strains were evaluated using sera from individual vaccine recipients. By determining the proportion of vaccinated subjects with functional bactericidal antibodies, assessment of the breadth of MenB-FHbp coverage at the individual level was determined, which is not possible using pooled sera. The 4 primary MenB test strains were selected to represent the diversity of MenB disease-causing IMD and thus support the potential breadth of coverage for MenB-FHbp using hSBA. Responses of individuals with hSBA titers ≥1:4 are the accepted correlate of protection and a surrogate of meningococcal vaccine efficacy. Thus, the responses provide a comprehensive and biologically predictive assessment of breadth of vaccine coverage. The relevance of the hSBA responses to the 4 primary MenB test strains to describe breadth of vaccine coverage is supported by the demonstration of protective bactericidal responses by MenB-FHbp also observed against diverse and contemporary MenB outbreak strains from Europe and the United States and against non-MenB disease-causing strains (ie, meningococcal serogroups C, Y, W, and X).

Another methodology, the enzyme-linked immunosorbent assay-based Meningococcal Antigen Typing System (MATS), has been used to predict vaccine coverage of MenB-4C. However, MATS only predicts coverage of antigens specific to MenB-4C and is not useful for assessing coverage of other vaccines with different antigen compositions. Specifically, MATS measures antigen expression rather than bactericidal activity and is reported as a relative potency compared with a reference strain for each antigen. If the relative potency for any one of the component antigens is commensurate with bactericidal activity for MenB-4C immune sera (ie, achieves a positive bactericidal threshold), the strain is considered susceptible to killing. However, because sera from vaccinated individuals are not used in MATS, the assay is unable to predict the proportion of a population achieving hSBA titers ≥1:4 (ie, the correlate of protection) in response to immunization.

Of note, limitations in performing hSBAs exist. For example, hSBAs are labor intensive and can require large quantities of sera and assay-compatible complement, particularly when larger numbers of strains and/or sera are to be assessed. In addition, interlaboratory differences in the performance of the assay reagents and strains used in hSBAs limit comparison of responses and assessments of breadth of coverage between vaccines. A known limitation of PPV analysis is the dependence of the magnitude of the response on prevalence (ie, in this setting, the proportion of subjects achieving hSBA ≥LLOQ for the additional strains). However, it is notable in this analysis that although there was a range of postvaccination responses to the additional strains (at 1 month postdose 2 and postdose 3), PPVs were uniformly high.

Taken together, the immunogenicity data obtained from the 10 additional MenB hSBA test strains support the response data obtained from the 4 primary MenB hSBA test strains and confirm the broad coverage of MenB isolates conferred by MenB-FHbp. This is the first work that has applied a rigorous assessment of a MenB vaccine's elicited immune response using the epidemiology of MenB strains with regard to the vaccine antigen sequence and expression, in conjunction with the recognized surrogate of protection (hSBA), and, using this knowledge, led to vaccine licensure.

Methods

Quantitation of FHbp Surface Expression

For all strains, FHbp surface expression was quantified by the MEASURE assay, a flow cytometric assay using monoclonal antibody (MN86-994-11) recognition of a conserved FHbp epitope common to both FHbp subfamilies. Details of the MEASURE assay have been described previously. The cutoff level adopted for each FHbp variant group was the observed median mean fluorescence intensity plus 1 standard deviation, using the precision estimate of 25.2% relative standard deviation.

Immunogenicity Analysis

Each of the 10 additional MenB test strains were used in hSBAs to test sera from subjects participating in 2 pivotal phase 3 studies of MenB-FHbp. A total of 900 subjects from each study were to be divided into 3 subsets (n=300 each); the 10 additional test strains were allocated across these subsets so that 2 subsets each included 3 test strains and 1 subset included 4 test strains. The subsets included samples from 300 subjects to ensure that 150 evaluable hSBA results from each study would be obtained. Immune responses measured by hSBA using phase 3 clinical study sera were based on the assay LLOQ, which was an hSBA titer equal to 1:8 or 1:16 depending on the strain.

Positive Predictive Value Analyses

The PPV for each primary/additional strain pair within an FHbp subfamily was defined as the proportion of subjects responding to the additional strain (hSBA titer ≥LLOQ for the additional strain) among the total number of primary strain responders (hSBA titer ≥LLOQ for the primary strain). PPV analyses assessed whether observed hSBA responses to the 4 primary strains predicted immune responses to additional strains expressing FHbps from the same subfamily.

1-40. (canceled)

41. A method of inducing an immune response in a human against Neisseria meningitidis serogroup B comprising administering an effective amount of a composition that comprises a) a first lipidated polypeptide comprising the amino acid sequence set forth in SEQ ID NO: 1, and b) a second lipidated polypeptide comprising the amino acid sequence set forth in SEQ ID NO: 2, wherein the composition induces an immune response against at least one N. meningitidis serogroup B strain expressing a polypeptide selected from the group consisting of A02, A28, A42, A63, A76, B05, B07, B08, B13, B52 and B107.

42. The method according to claim 41, wherein the immune response induced is bactericidal.

43. The method according to claim 42, wherein the composition further comprises polysorbate-80.

44. The method according to claim 43, wherein the composition further comprises aluminum.

45. The method according to claim 44, wherein the composition further comprises histidine buffer.

46. The method according to claim 45, wherein the composition further comprises sodium chloride.

47. The method according to claim 46, wherein the composition comprises about 120 μg/mL of the first polypeptide; about 120 μg/mL of the second polypeptide; about 2.8 molar ratio of polysorbate-80; about 0.5 mg/mL aluminum; about 10 mM histidine; and about 150 mM sodium chloride.

48. The method according to claim 46, wherein the composition comprises about 60 μg of the first polypeptide; about 60 μg of the second polypeptide; about 18 μg polysorbate-80; about 250 μg aluminum; about 780 μg histidine; and about 4380 μg sodium chloride.

49. The method according to claim 41, wherein the composition further comprises at least one additional immunogenic composition comprising a mixture of four distinct and separately made protein-capsular polysaccharide conjugates, wherein the first conjugate comprises N. meningitidis capsular polysaccharide of serogroup W conjugated to a carrier protein, the second conjugate comprises N. meningitidis capsular polysaccharide of serogroup Y conjugated to a carrier protein, the third conjugate comprises N. meningitidis capsular polysaccharide of serogroup A conjugated to a carrier protein, and the fourth conjugate comprises N. meningitidis capsular polysaccharide of serogroup C conjugated to a carrier protein, wherein the carrier protein in each of the four conjugates is independently selected from the group consisting of diphtheria toxoid, CRM₁₉₇, and tetanus toxoid.

50. The method according to claim 49, wherein the composition induces an immune response against at least one Neisseria meningitidis serogroup A strain.

51. The method according to claim 49, wherein the composition induces an immune response against at least one Neisseria meningitidis serogroup C strain.

52. The method according to claim 49, wherein the composition induces an immune response against at least one Neisseria meningitidis serogroup W strain.

53. The method according to claim 49, wherein the composition induces an immune response against at least one Neisseria meningitidis serogroup Y strain.

54. The method according to claim 41, wherein the effective amount of the composition comprises one dose.

55. The method according to claim 41, wherein the effective amount of the composition comprises two doses.

56. The method according to claim 55, wherein the effective amount of the composition further comprises a booster dose.

57. The method according to claim 41, wherein the composition does not comprise a hybrid or a fusion protein.

58. The method according to claim 49, wherein the Neisseria meningitidis serogroup A (MenA) capsular saccharide is conjugated to an adipic acid dihydrazide (ADH) linker by 1-cyano-4-dimethylamino pyridinium tetrafluoroborate chemistry, wherein the linker is conjugated to tetanus toxoid carrier protein (TT) by carbodiimide chemistry (MenA_{AH}-TT conjugate); the Neisseria meningitidis serogroup C (MenC) capsular saccharide is conjugated to an ADH linker by 1-cyano-4-dimethylamino pyridinium tetrafluoroborate chemistry, wherein the linker is conjugated to tetanus toxoid carrier protein (TT) by carbodiimide chemistry (MenC_{AH}-TT conjugate); the Neisseria meningitidis serogroup W (MenW) capsular saccharide is directly conjugated to tetanus toxoid carrier protein (TT) by 1-cyano-4-dimethylamino pyridinium tetrafluoroborate chemistry, in the absence of a linker (MenW-TT conjugate); and the Neisseria meningitidis serogroup Y (MenY) capsular saccharide is directly conjugated to tetanus toxoid carrier protein (TT) by 1-cyano-4-dimethylamino pyridinium tetrafluoroborate chemistry, in the absence of a linker (MenY-TT conjugate).

59. The method according to claim 41, wherein the composition induces a bactericidal titer of serum immunoglobulin that is at least 2-fold higher in the human after receiving a first dose than a bactericidal titer of serum immunoglobulin in the human prior to receiving the first dose, when measured under identical conditions in a serum bactericidal assay using human complement.
